(19) United States
(12) Patent Application Publication
(10) Pub. No.: US 2014/0287023 A1
(43) Pub. Date: Sep. 25, 2014

(54) 5'-TRIPHOSPHATE OLIGORIBONUCLEOTIDES

(71) Applicants: McGill University, Montreal (CA); Oregon Health & Science University, Portland, OR (US)

(72) Inventors: John Hiscott, Singer Island, FL (US); Rongtuan Lin, Montreal (CA); Meztli Arguello, Montreal (CA)

(21) Appl. No.: 14/177,866

(22) Filed: Feb. 11, 2014

Related U.S. Application Data
(60) Provisional application No. 61/763,367, filed on Feb. 11, 2013.

(51) Int. Cl. C12N 15/11

(52) U.S. Cl. CPC C12N 15/11
USPC 424/450; 536/23.1; 514/44 R

(57) ABSTRACT

5′-triposphate oligoribonucleotides, pharmaceutical compositions comprising said 5′-triposphate oligoribonucleotides, and methods of using said 5′-triposphate oligoribonucleotides to treat viral infections are disclosed.

### PRIORITY CLAIM

This application claims the benefit of U.S. Provisional Application 61/763,367, filed 11 Feb. 2013 and is hereby incorporated by reference in its entirety.

### FIELD

Generally, the field is RNA-based therapeutic molecules. More specifically, the field is 5′-triposhpate oligoribonucleotide immune system agonists and pharmaceutical compositions comprising the same.

### BACKGROUND

The innate immune system has evolved numerous molecular sensors and signaling pathways to detect, contain and clear viral infections (Takeuchi O and Akira S Immunol Rev 227, 75-86 (2009); Yoneyama M and Fujita T, Rev Med Virol 20, 4-22 (2010); Wilkins C and Gale M Curr Opin Immunol 22, 41-47 (2010); and Brennan K and Bowie A G Curr Opin Microbiol 13, 503-507 (2010); all of which are incorporated by reference herein.) Viruses are sensed by a subset of pattern recognition receptors (PRRs) that recognize evolutionarily conserved structures known as pathogen-associated molecular patterns (PAMPs). Classically, viral nucleic acids are the predominant PAMPs detected by these receptors during infection. These sensing steps contribute to the activation of signaling cascades that culminate in the early production of antiviral effector molecules, cytokines and chemokines responsible for the inhibition of viral replication and the induction of adaptive immune responses (Takeuchi O and Akira S Cell 140, 805-820 (2010), Liu S Y et al, Curr Opin Immunol 23, 57-64 (2011); and Akira S et al, Cell 124, 783-801 (2006); all of which are incorporated by reference herein). In addition to the nucleic acid sensing by a subset of endosome-associated Toll-like receptors (TLR), viral RNA structures within the cytoplasm are recognized by members of the retinoic acid-inducible gene-I (RIG-I)-like receptors (RLRs) family, including the three DExD/H box RNA helicases RIG-I, Mda5 and LGP-2 (Kumar H et al, Int Rev Immunol 30, 16-34 (2011); Loo Y M and Gale M, Immunity 34, 680-692 (2011); Belgnaoui S M et al, Curr Opin Immunol 23, 564-572 (2011); Beutler B E, Blood 113, 1399-1407 (2009); Kawai T and Akira S, Immunity 34, 637-650 (2011); all of which are incorporated by reference herein.)

RIG-I is a cytosolic multidomain protein that detects viral RNA through its helicase domain (Jiang F et al, Nature 479, 423-427 (2011) and Yoneyama M and Fujita T, J Biol Chem 282, 15315-15318 (2007); both of which are incorporated by reference herein). In addition to its RNA sensing domain, RIG-I also possesses an effector caspase activation and recruitment domain (CARD) that interacts with the mitochondrial adaptor MAVS, also known as VISA, IPS-1, and Cardif (Kawai T et al, Nat Immunol 6, 981-988 (2005) and Meylan E et al, Nature 437, 1167-1172 (2005), both of which are incorporated by reference herein.) Viral RNA binding alters RIG-I conformation from an auto-inhibitory state to an open conformation exposing the CARD domain, resulting in RIG-I activation which is characterized by ATP hydrolysis and ATP-driven translocation of RNA (Schlee M et al, Immunity 31, 25-34 (2009); Kowlinski E et al, Cell 147, 423-435 (2011); and Myong S et al, Science 323, 1070-1074 (2011); all of which are incorporated by reference herein). Activation of RIG-I also allows ubiquitination and/or binding to polyubiquitin. In recent studies, polyubiquitin binding has been shown to induce the formation of RIG-I tetramers that activate downstream signaling by inducing the formation of prion-like fibrils comprising the MAVS adaptor (Jiang X et al, Immunity 36, 959-973 (2012); incorporated by reference herein). MAVS then triggers the activation of IRF3, IRF7 and NF-κB through the IKK-related serine kinases TBK1 and IKKE (Sharma S et al, Science 300, 1148-1151 (2003); Xu L G et al, Molecular Cell 19, 727-740 (2005); and Seth R B et al, Cell 122, 669-682 (2005); all of which are incorporated by reference herein). This in turn leads to the expression of type I interferons (IFNβ and IFNα), as well as pro-inflammatory cytokines and anti-viral factors (Tamassia N et al, J Immunol 181, 6563-6573 (2008) and Kawai T and Akira S, Ann NY Acad Sci 1143, 1-20 (2008); both of which are incorporated by reference herein.) A secondary response involving the induction of IFN stimulated genes (ISGs) is induced by the binding of IFN to its cognate receptor (IFNα/βR). This triggers the JAK-STAT pathway to amplify the antiviral immune response (Wang B X and Fish E N Trends Immunol 33, 190-197 (2012); Nakhaei P et al, Activation of Interferon Gene Expression Through Toll-like Receptor-dependent and -independent Pathways, in The Interferons, Wiley-VCH Verlag GmbH and Co KGaA, Weinheim FRG (2006); Sadler A J and Wiliams B R, Nat Rev Immunol 8, 559-568 (2008); and Schoggins J W et al, Nature 472, 481-485 (2011); all of which are incorporated by reference herein.)

The nature of the ligand recognized by RIG-I has been the subject of intense study given that PAMPs are the initial triggers of the antiviral immune response. In vitro synthesized RNA carrying an exposed 5′ terminal triphosphate (5′ ppp) moiety was identified as a RIG-I agonist (Hornung V et al, Science 314, 994-997 (2006); Pichlmair A et al, Science 314, 997-1001 (2006); and Kim D H et al, Nat Biotechol 22, 321-325 (2004); all of which are incorporated by reference herein). The 5′ ppp moiety is added to the end of all viral and eukaryotic RNA molecules generated by RNA polymerization. However, in eukaryotic cells, RNA processing in the nucleus cleaves the 5′ ppp end and the RNA is capped prior to release into the cytoplasm. The eukaryotic immune system evolved the ability to distinguish viral ‘non-self’ 5′ ppp RNA from cellular ‘self’ RNA through RIG-I (Fujita T, Immunity 31, 4-5 (2009); incorporated by reference herein). Further characterization of RIG-I ligand structure indicated that blunt base pairing at the 5′ end of the RNA and a minimum double strand (ds) length of 20 nucleotides were also important for RIG-I signaling (Schlee M and G Hartmann, Molecular Therapy 18, 1254-1262 (2010); incorporated by reference herein). Further studies indicated that a dsRNA length of less than 300 base pairs led to RIG-I activation but a dsRNA length of more than 2000 bp lacking a 5′ ppp (as is the case with poly I:C) failed to activate RIG-I. (Kato H et al, J Exp Med 205, 1601-1610 (2008); incorporated by reference herein).

RNA extracted from virally infected cells, specifically viral RNA genomes or viral replicative intermediates, was also shown to activate RIG-I (Baum A et al, Proc Natl Acad Sci USA 107, 16303-16308 (2010); Rehwinkel J and Sousa C R E, Science 327, 284-286 (2010); and Rehwinkel J et al, Cell 140, 397-408 (2010); all of which are incorporated by reference herein). Interestingly, the highly conserved 5′ and 3′ untranslated regions (UTRs) of negative single strand RNA virus genomes display high base pair complementarity and the panhandle structure theoretically formed by the viral genome meets the requirements for RIG-I recognition. The elucidation of the crystal structure of RIG-I highlighted the molecular interactions between RIG-1 and 5′ppp-dsRNA (Cui S et al, Molecular Cell 29, 169-179 (2008); incorporated by reference herein), providing a structural basis for the conformational changes involved in exposing the CARD domain for effective downstream signaling.

### SUMMARY

Disclosed herein is a oligoribonucleotide derived from the 5′ and 3′UTRs of the VSV genome (SEQ ID NO: 1) synthesized with a triphosphate group at its 5′ end (5′ppp-SEQ ID NO: 1). The 5′ppp-SEQ ID NO: 1 activates the RIG-I signaling pathway and triggers a robust antiviral response that interferes with infection by several pathogenic viruses, including Dengue, HCV, HIV-1 and H1N1 Influenza A/PR/8/34. Furthermore, intravenous delivery of 5′ppp-SEQ ID NO: 1 stimulates an antiviral state in vivo that protects mice from lethal influenza virus challenge.

Also disclosed are modified variants of 5′ppp-SEQ ID NO: 1 that include locked nucleic acids, G-clamp nucleotides, nucleotide base analogs, terminal cap moieties, phosphate backbone modifications, conjugates, and the like.

Also disclosed are pharmaceutical compositions comprising 5′ppp-SEQ ID NO: 1 and/or a modified variant thereof and a pharmaceutically acceptable carrier that acts as a transfection reagent such as a lipid based carrier, a polymer based carrier, a cyclodextrin based carrier, a protein based carrier and the like.

Also disclosed are methods of treating a viral infection in a subject by administering one or more of the pharmaceutical compositions to a subject.

### BRIEF DESCRIPTION OF THE SEVERAL VIEWS OF THE DRAWINGS

The term “5′ pppRNA,” used in the figures is equivalent to the term “5′ppp-SEQ ID NO: 1” used in the text and may be used interchangeably.

FIG. 1A through FIG. 1D show that 5′ppp-SEQ ID NO: 1 stimulates an antiviral and inflammatory response in lung epithelial A549 cells.

FIG. 1A is a 2-D representation of 5′ppp-SEQ ID NO: 1 (top panel) and an image of a gel showing that the in vitro transcription product of 5′-ppp-SEQ ID NO: 1 is a single product degraded by RNAse I.

FIG. 1B is an image of an immunoblot in which 5′ppp-SEQ ID NO: 1 or a homologous control of SEQ ID NO: 1 alone (lacking the 5′-triphosphate) was mixed with Lipofectamine RNAiMax® and transfected at the RNA concentrations indicated (0.1-500 ng/ml) into A549 cells. At 8 hours post treatment, whole cell extracts were prepared, resolved by SDS-page and immunoblotted with antibodies specific for IRF3 pSer396, IRF3, ISG56, NOXA, cleaved caspase 3, PARP and β-actin as indicated. Results are from a representative experiment; all immunoblots are from the same samples.

FIG. 1C is an image of immunoblots of whole cell extracts of A549 cells transfected with 10 ng/ml 5′ppp-SEQ ID NO: 1 and probed with antibodies specific to the indicated proteins. Whole cell extracts were prepared at different times after transfection (0-48 hours), electrophoresed by SDS-PAGE and probed with antibodies specific for IRF3 pSer-396, IRF3, IRF7, STAT1 pTyr-701, STAT1, ISG56, RIG-I, IκBα pSer-32, IkBα and β-actin. All immunoblots are from the same samples. To detect IRF3 dimerization (top panel,) whole cell extracts were resolved by native-PAGE and analyzed by immunoblotting for IRF3.

FIG. 1D is a set of two bar graphs showing the results of ELISA assays to detect IFNβ and IFNα in cell culture supernatants at the indicated times. Error bars represent SEM from two independent samples.

FIGS. 2A-2D demonstrate that the induction of the interferon response by 5′ppp-SEQ ID NO: 1 is dependent on functional RIG-I signaling

FIG. 2A is a set of two bar graphs showing the fold induction of IFNβ and IFNα4 in wild type and RIG-I^{-/-} mouse endothelial fibroblasts (MEF's) by 5′ppp-SEQ ID NO: 1 and a constitutively active form of RIG-I (ΔRIG-I) (100 ng). MEF's were co-transfected with an IFNα4 or IFNβ promoter reporter plasmid (200 ng) along with 5′ppp-SEQ ID NO: 1 (500 ng/ml) or an expression plasmids encoding ΔRIG-I. An IRF-7 expression plasmid (100 ng) was added for transactivation of the IFNα4 promoter. Luciferase activity was analyzed 24 hours post transfection by the Dual-Luciferase Reporter assay. Relative luciferase activity was measured as fold induction relative to the basal level of reporter gene. Error bars represent SEM from nine replicates performed in three independent experiments.

FIG. 2B is a bar graph showing the induction of IFNβ in MDA5^{-/-}, TLR3^{-/-}, TLR7^{-/-} and RIG-I^{-/-} MEFs by 5′ppp-SEQ ID NO: 1 and ΔRIG-I. MEFs were co-transfected with IFNβ promoter reporter plasmid (200 ng) along with 5′ppp-SEQ ID NO: 1 (500 ng/ml). Luciferase activity was analyzed 24 h post-transfection by the Dual-Luciferase Reporter assay. Relative luciferase activity was measured as fold induction relative to the basal level of reporter gene. Promoter activity in the knockout MEFs was then normalized against the activity in their respective wild type MEF's to obtain the percentage of activation. Error bars represent SEM from nine replicates performed in three independent experiments.

FIG. 2C is an image of a set of immunoblots of whole cell extracts of A549 cells and A549 cells deficient in MAVS expression. 5′ppp-SEQ ID NO: 1 was transfected in control A549 and MAVS shRNA A549 cells at different concentrations (0, 0.1, 1, 10, 100 ng/ml). At 8 hours after treatment, whole cell extracts were analyzed by SDS-PAGE, blotted, and probed with antibodies specific for pIRF3 Ser-396, IRF3, pSTAT1 Tyr 701, STAT1, ISG56, MAVS (VISA), and β-Actin. Results are from a representative experiment; all immunoblots are from the same samples.

FIG. 2D is an image of an immunoblot of whole cell extracts of A549 cells, A549 cells transfected with siRNA that silences RIG-I expression, and an irrelevant negative control siRNA. Cells were transfected with 5′-ppp-SEQ ID NO: 1 as indicated and whole cell extracts were analyzed by SDS-PAGE, blotted, and probed with antibodies specific for the indicated proteins.

FIGS. 3A-3E depict 5′ppp-SEQ ID NO: 1 acting as a broad-spectrum antiviral agent.

FIG. 3A is a set of three bar graphs showing the percent of cells infected with VSV, Dengue, and Vaccina as indicated and treated with 5′ppp-SEQ ID NO: 1 as indicated. A549 cells were transfected with 10 ng/ml 5′ppp-SEQ ID NO: 124 hours prior to infection and infected with VSVΔ51-GFP (MOI=0.1), Dengue virus (MOI=0.1), and Vaccinia-GFP virus (MOI=5), respectively. Percentage of infected cells was determined 24 hours post-infection by flow cytometry analysis of GFP expression (VSV-GFP and Vaccinia-GFP) or intracellular staining of DENV E protein expression (Dengue virus). Data are from a representative experiment performed in triplicate. Error bars represent the standard deviation.

FIG. 3B is a set of six flow cytometry plots showing the results of CD14⁺ and CD14⁻ human PBMCs treated with 5′ppp-SEQ ID NO: 1 as indicated and infected with Dengue virus as indicated. PBMCs were transfected with 100 ng/ml 5′ppp-SEQ ID NO: 124 hours prior to infection with dengue virus at an MOI of 5. At 24 hours post-infection, the percentage of Dengue infected CD14⁺ and CD14⁻ cells was evaluated by intracellular staining of DENV E protein expression by flow cytometry. Data are from a representative experiment performed in triplicate. Error bars represent the standard deviation.

FIG. 3C is a bar graph showing the results of human PBMC's infected with DENV2 as indicated, treated with 5′ppp-SEQ ID NO: 1 (called 5′ pppVSV in this figure), and treated with the Lyovec® transfection agent as indicated. Human PBMCs from three different donors were transfected with 100 ng/ml 5′ppp-SEQ ID NO: 1 prior to infection with Dengue virus at an MOI of 5. The percentage of Dengue infected cells in the CD14⁺ population was evaluated by intracellular staining of DENV E protein expression using flow cytometry. Data are from an experiment performed in triplicate on three different patients. Error bars represent the standard deviation.

FIG. 3D is a set of three flow cytometry histograms depicting the results of human CD4⁺ T cells infected with HIV-GMP as indicated and treated with 5′ppp-SEQ ID NO: 1 as indicated. CD4⁺ T cells were isolated from human PBMCs and activated with anti-CD3 and anti-CD28 antibodies. Cells were incubated in the presence or absence of supernatant from 5′ppp-SEQ ID NO: 1-treated monocytes for 4 hours and infected with HIV-GFP (MOI=0.1) for 48 hours. The percentage of HIV infected, activated CD4⁺ T cells (GFP positive) was assessed by flow cytometry.

FIG. 3E is an image of an immunoblot of whole cell extracts of Huh7 and Huh7.5 cells transfected with 5′ppp-SEQ ID NO: 1 (10 ng/ml) as indicated and infected with Hepatitis C Virus (HCV) 24 hours after treatment with 5′ppp-SEQ ID NO: 1 as indicated. At 48 hours post-infection, analyzed by SDS-PAGE, blotted, and probed with antibodies specific for the HCV viral protein NS3 and IFIT1 as well as β-actin.

FIGS. 4A-4F depict 5′ppp-SEQ ID NO: 1 as an inhibitor of H1N1 Influenza replication in vitro.

FIG. 4A is an image of an immunoblot of whole cell extracts from A549 cells probed with antibodies to the indicated proteins. A549 cells were treated with 5′ppp-SEQ ID NO: 1 (10 ng/ml) as indicated. At 24 hours post-treatment, cells were infected with an increasing MOI of A/PR8/34 H1N1 Influenza virus (0.02 MOI, 0.2 MOI, or 2 MOI) for 24 hours. Whole cell extracts were run on an SDS-PAGE gel and immunoblotted to detect expression of the influenza viral proteins NS1, ISG56, and β-actin.

FIG. 4B is a bar graph depicting viral titers in the cell culture supernatants from the samples shown in FIG. 7A. Viral titer was determined by plaque assay. Error bars represent the standard error of the mean from two independent samples.

FIG. 4C is an image of an immunoblot of whole cell extracts of A549 cells probed with antibodies to the indicated proteins. A549 cells were treated with increasing concentrations of 5′ppp-SEQ ID NO: 1 (0.1 ng/ml to 10 ng/ml) for 24 hours prior to infection with 0.2 MOI of influenza. Whole cell extracts were run on an SDS-PAGE gel and immunoblotted to detect expression of viral proteins NS1, ISG56, and β-Actin.

FIG. 4D is a bar graph depicting the viral titers in cell culture supernatants from the samples shown in FIG. 6C. Viral titer was determined by plaque assay. Error bars represent SEM from two independent samples.

FIG. 4E is an image of an immunoblot of whole cell extracts of A549 cells probed with antibodies to the indicated proteins. A549 cells were treated with 5′ppp-SEQ ID NO: 1 (10 ng/ml) both before and after infection with 0.02 MOI of influenza as indicated on the legend above the gel (numbers are in days.) Whole cell extracts were run on an SDS-PAGE gel and immunoblotted to detect expression of the indicated proteins.

FIG. 4F is an image of an immunoblot of whole cell extracts of A549 cells transfected with a control siRNA, RIG-I siRNA or IFNα/β receptor siRNA and then treated with 5′-ppp-SEQ ID NO: 1 at 10 ng/ml as indicated and infected with Influenza at 0.2 MOI as indicated. The whole cell extracts were prepared 24 hours after infection, run on an SDS-PAGE gel, and immunoblotted to detect expression the indicated proteins.

FIG. 4G is an immunoblot of whole cell extracts of A549 cells transfected with a control siRNA or an IFNα/βR siRNA and then treated with 5′-ppp-SEQ ID NO: 1 at 10 ng/ml or IFNα-2b at 100 IU/ml) for 24 hours. The whole cell extracts were prepared 24 hours after infection, run on an SDS-PAGE gel, and immunoblotted to detect expression the indicated proteins.

FIGS. 5A-5I demonstrate that 5′ppp-SEQ ID NO: 1 activates innate immunity and protects mice from lethal influenza infection in vivo. All mice treated with 5′ppp-SEQ ID NO: 1 were injected intravenously with 25 μg of 5′ppp-SEQ ID NO: 1 in complex with In vivo Jet-PEI®. Statistical analysis was performed by Student's t test (*, p≦0.05; **, p≦0.01; ***, p≦0.001; ns, not statistically significant).

FIG. 5A is a plot depicting the percent survival over time of mice treated with 5′ppp-SEQ ID NO: 1 one day prior to infection with 500 PFU of influenza relative to non-treated (NT) mice as indicated.

FIG. 5B is a plot depicting the percent weight loss over time of mice treated with 5′ppp-SEQ ID NO: 1 one day prior to infection with 500 PFU of influenza relative to non-treated (NT) mice as indicated.

FIG. 5C is a bar graph depicting the influenza viral titer over time in the lung of mice treated with 5′ppp-SEQ ID NO: 1 one day prior to infection with 500 PFU of influenza relative to non-treated (NT) mice as indicated. Viral titer was measured by plaque assay. Error bars represent the SEM from six animals. ND: not detected.

FIG. 5D is a bar graph depicting the influenza viral titer at 3 days after infection in mice treated with 5′ppp-SEQ ID NO: 1 one day prior to and on the day of infection with 500 PFU of influenza; one day prior to, on the day of, and one day following the day of infection with 5′ppp-SEQ ID NO: 1; and mice infected with 500 PFU of influenza but otherwise untreated (NT). Viral titer was determined by plaque assay. Error bars represent the SEM from five different animals.

FIG. 5E is a bar graph depicting the influenza viral titer in mice infected with 50 PFU of influenza on day 0 and treated with 5′ppp-SEQ ID NO: 1 on day −1 and day 0 (prophylactic), or on day 1 and day 2 (therapeutic). Lung viral titers were determined on Day 3. Error bars represent the standard error of the mean from five animals.

FIG. 5F is a bar graph depicting the results of an ELISA assay for serum IFNβ in wild type, TLR3^{-/-}, and MAVS^{-/-} mice as indicated. All mice were treated with 5′ppp-SEQ ID NO: 1. IFNβ was quantified by ELISA 6 hours. Error bars represent the standard error of the mean from three animals.

FIG. 5G is a bar graph depicting the results of wild type and MAVS^{-/-} mice treated with 5′ppp-SEQ ID NO: 1 as indicated and infected with influenza at 500 PFU. Lungs were collected and homogenized on Day 1 and lung viral titers were determined by plaque assay. Error bars represent the standard error of the mean from four different animals.

FIG. 5H is a line plot showing survival of IFNα/βR^{-/-} mice treated with 5′ppp-SEQ ID NO: 1 as indicated and infected with influenza at 100 PFU. Survival was monitored for 18 days.

FIG. 5I is a bar graph depicting the results of an ELISA assay for serum IFNβ in mice treated with 5′ppp-SEQ ID NO: 1 and non-treated (NT) mice. Serum was collected 6 hours after treatment. Error bars represent the SEM from three animals.

FIGS. 6A-6C demonstrate that 5′ppp-SEQ ID NO: 1 treatment controls influenza-mediated pneumonia.

FIG. 6A is an image of representative lung samples from the following groups: In the far left panels animals were treated with neither 5′ppp-SEQ ID NO: 1 nor infected with influenza. In the panels second from left, animals were treated with 5′ppp-SEQ ID NO: 1, but not infected with influenza. In the panels second from right, animals were infected with influenza but not treated with 5′ppp-SEQ ID NO: 1. In the panels on the right, animals were infected with influenza and treated with 5′ppp-SEQ ID NO: 1. Lungs were collected on day 3 and day 8 post-infection and stained with hematoxylin and eosin (H&E). The images in FIG. 9A highlight inflammation and tissue damage.

FIG. 6B is an image of representative lung samples of influenza infected animals either treated with 5′ppp-SEQ ID NO: 1 (top panel) or untreated (bottom panel) highlighting the extent of pneumonia.

FIG. 6C is a bar graph summarizing inflammation, tissue damage and surface area affected by pneumonia of the groups described in the legend for FIG. 9A as scored by a veterinary pathologist. Grade 1=nil; Grade 2=modest, rare; Grade 3=moderate, frequent; Grade 4=severe, extensive.

FIG. 8A (left panel) is a bar graph depicting the VSV virus titer from the supernatants from the experiment described in FIG. 6A was determined by standard plaque assay. The right panel is an image of an immunoblot probed with antibodies specific for VSV proteins.

FIG. 8B is a set of two bar graphs depicting the dengue virus titer from supernatants described in FIG. 6A determined by plaque assay (left panel) and the virus titer from the supernatants using primers specific for Dengue RNA (SEQ ID NO: 29 and SEQ ID NO: 30.)

FIG. 9A is a set of four bar graphs depicting IFNα and IFNβ protein expression in the serum and lung homogenates of mice treated with 25 μg of 5′ppp-SEQ ID NO: 1 in complex with In vivo Jet-PEI™. Protein expression was determined by ELISA at the indicated time post treatment. Error bars represent the standard error of the mean from three animals.

FIG. 9B is a set of four bar graphs depicting RIG-I and IFIT1 RNA expression in spleen and lung homogenates of mice treated with 25 μg of 5′ppp-SEQ ID NO: 1 in complex with In vivo Jet-PEI™. RNA expression was determined by RT-PCR at the indicated time post treatment. Error bars represent the standard error of the mean from three animals.

FIG. 9C is a set of three bar graphs depicting the indicated cellular populations in lung homogenates of mice treated with 25 μg of 5′ppp-SEQ ID NO: 1 in complex with In vivo Jet-PEI™. Lungs were minced and digested with collagenase IV and DNAse I for 30 minutes, mixed for 15 minutes, and then filtered through a 70 μM nylon filter. Cell types were analyzed by flow cytometry and the values given relative to CD45⁺ leukocytes. Error bars represent the standard error of the mean from four animals.

FIG. 9D is a set of four bar graphs depicting CXCL10 and IRF7 RNA expression in spleen (left) and lung (right) homogenates of mice treated with 25 μg of 5′ppp-SEQ ID NO: 1 in complex with In vivo Jet-PEI™. RNA expression was determined by RT-PCR at the indicated time post treatment. Error bars represent the standard error of the mean from three animals.

FIG. 10A is a set of six flow cytometry plots showing infection of A549 cells with Dengue Virus (DENV) with and without 5′ppp-SEQ ID NO: 1.

FIG. 10B is a bar graph summarizing flow cytometry data of infection of A549 cells in the presence of the indicated concentration of 5′ppp-SEQ ID NO: 1 or a negative control RNA.

For both FIGS. 10A and 10B, A549 cells were pretreated with various concentrations of 5′ppp-SEQ ID NO: 1 (0.01 to 10 ng/ml) or control (Ctrl) RNA lacking the 5′ ppp at the same concentrations for 24 h prior to DENV challenge. The percentage of DENV-infected cells was determined by intracellular staining (ICS) of DENV E protein expression using flow cytometry. Data are from two independent experiments performed in triplicate and represent the means SEM. *, P<0.05. FSC, forward scatter.

FIG. 10C is a bar graph showing DENV RNA expression in DENV infected cells according to the indicated conditions.

FIG. 10D is a bar graph showing viral titer and image of a Western blot showing DENV protein expression in DENV infected cells according to the indicated conditions.

For FIGS. 10C and 10D, A549 cells were pretreated with 5′ppp-SEQ ID NO: 1 (1 ng/ml) for 24 h prior to DENV challenge (MOI, 0.1). DENV RNA level (FIG. 10C), viral titers (FIG. 10D), and DENV E protein expression level (FIG. 10D) were determined by RT-qPCR, plaque assay, and Western blotting, respectively. Error bars represent SEM from three independent samples. *, P<0.05. One representative DENV E protein Western blot out of three independent triplicates is shown.

FIG. 10E is a bar graph showing DENV E protein expression in A549 cells infected according to the indicated conditions. A549 cells were transfected using Lipofectamine (Lipo.) RNAiMax with increasing concentrations of 5′ppp-SEQ ID NO: 1 and poly(I:C) (0.1 to 1 ng/ml) or treated with the same dsRNA sequences (5,000 ng/ml) in the absence of transfection reagent. Cells were then challenged with DENV (MOI, 1), and the percentage of infected cells was determined by FACS 24 h after infection. Data are the means±SEM from two independent experiments performed in triplicate. *, P 0.05.

FIG. 10F is a bar graph showing DENV E protein expression in A549 cells infected according to the indicated conditions.

FIG. 10G is a bar graph showing cell viability in A549 cells treated as indicated. The percentage of A549 DENV-infected cells and cell viability were assessed by flow cytometry and determined at 24 h (black bars), 48 h (gray bars), and 72 h (white bars) after DENV challenge (MOI, 0.01). Cells were pretreated with 5′ppp-SEQ ID NO: 1 (1 ng/ml) for 24 h before DENV challenge. Data are the means±SEM from a representative experiment performed in triplicate. *, P<0.05.

FIG. 11A is a bar graph of DENV E protein expression in A549 cells treated according to the indicated conditions. A549 cells were treated with 5′ppp-SEQ ID NO: 1 (1 ng/ml) 4 h (black bars) or 8 h (gray bars) following DENV challenge (MOI, 0.01). The percentage of DENV-infected cells was determined by intracellular staining (ICS) of DENV E protein expression using flow cytometry at 48 h after infection. Data represent the means±SEM from a representative experiment performed in triplicate. *, P<0.05.

FIG. 11B is a bar graph of DENV RNA expression in A549 cells treated according to the indicated conditions. DENV RNA levels were determined by RT-qPCR (48 h after infection) on A549 cells treated with 5=pppRNA (1 ng/ml) 4 h (black bars) and 8 h (gray bars) after infection. *, P<0.05.

FIG. 11C is a bar graph summarizing flow cytometry indicating the viability of A549 cells treated according to the indicated conditions. Cell viability of A549 cells was measured by flow cytometry 24 h (black bars) and 48 h (gray bars) after infection. Cells were treated with 5′ppp-SEQ ID NO: 14 h after DENV infection. Data are the means±SEM from a representative experiment performed in triplicate.

FIG. 11D is an image of a western blot indicating expression of the indicated proteins in A549 cells treated according to the indicated conditions. A549 cells were challenged with DENV (MOI, 0.1) for 4 h and transfected with 5′ppp-SEQ ID NO: 1 (0.1 to 10 ng/ml) and incubated for an additional 20 h. Whole-cell extracts (WCEs) were prepared and subjected to immunoblot analysis 24 h postinfection. Data are from one representative experiment.

FIG. 11E is a set of four bar graphs indicating expression of the indicated genes in A549 cells treated according to the indicated conditions. A549 cells were infected with DENV at different MOI and were transfected with 5′ppp-SEQ ID NO: 1 (1 ng/ml) 4 h after infection. The expression level of genes was determined by RT-qPCR 24 h after DENV challenge. Data are the means±SEM from a representative experiment performed in triplicate. *, P<0.05.

FIG. 12A is an image of a western blot indicating the expression of the indicated proteins in A549 cells treated according to the indicated conditions. A549 cells were transfected with control or RIG-I siRNA (10 or 30 pmol), and 48 h later they were treated with 5′ppp-SEQ ID NO: 1 (10 ng/ml) for 24 h. Expression of IFIT1, RIG-I, and β-actin was evaluated by Western blotting. RIG-I knockdown and impairment of the 5′ppp-SEQ ID NO: 1-induced immune response is representative of at least 3 independent experiments.

FIG. 12B is a set of four bar graphs indicating the expression of the indicated genes in A549 cells treated according to the indicated conditions. A549 cells were transfected with control siRNA or RIG-I siRNA (30 pmol), and 48 h later they were treated with 5′ppp-SEQ ID NO: 1 (10 ng/ml) for 24 hours. mRNA expression level of IFN-α, IFN-β, TNF-α, and IL-29 was evaluated by RT-qPCR. Data are from a representative experiment performed in triplicate and show the means±SEM. *, P<0.05.

FIG. 12C is a bar graph of indicating the expression of DENV E protein in A549 cells treated according to the indicated conditions. A549 cells were transfected with control (black bars), RIG-I (gray bars), or a combination of TLR3/MDA5 (white bars) siRNA (30 pmol each), and 48 h later they were treated with 5′ppp-SEQ ID NO: 1 (10 ng/ml) or poly(I:C) (1 ng/ml). Cells were then infected with DENV (MOI, 0.5), and at 24 h p.i. the percentage of infected cells was assessed by intracellular staining of DENV E protein using flow cytometry. Data are from a representative experiment performed in triplicate and show the means±SEM. *, P<0.05.

FIG. 12D is a bar graph indicating the expression of DENV E protein in A549 cells treated according to the indicated conditions.

FIG. 12E is a bar graph indicating the expression of DENV E protein in A549 cells treated according to the indicated conditions.

For both FIGS. 12D and 12E: A549 cells were treated with 5′ppp-SEQ ID NO: 1 (0.1 to 10 ng/ml) for 24 h 2 days after transfection with 30 pmol of control (black bars), RIG-I (gray bars), or STING (white bars) siRNA (FIG. 12D) or with 30 pmol of control (black bars) or MAVS (gray bars) siRNA (FIG. 12E). Cells were then challenged with DENV (MOI, 0.1) for 24 h. The percentage of DENV-infected cells was determined by intracellular staining of DENV E protein and flow cytometry 24 h after infection. Data are the means±SEM from a representative experiment performed in triplicate. *, P<0.05.

FIG. 12F is a bar graph indicating the expression of DENV E protein in A549 cells treated according to the indicated conditions. TBK1^{+/+} (black bars) and TBK1^{-/-} (gray bars) MEF cells were treated with 10 ng/ml of 5′ppp-SEQ ID NO: 124 h before DENV challenge at an MOI of 5. The percentage of DENV-infected cells was evaluated by flow cytometry. Data are the means±SEM of a representative experiment performed in triplicate. *, P<0.05.

FIG. 13A is a set of three bar graphs indicating the expression of the indicated genes in A549 treated according to the indicated conditions. A549 cells were transfected with control, IFN-α/βRα chain (IFNAR1), IFN-α/βRβ chain (IFNAR2), or IL-28R siRNA, and 48 h later mRNA levels of IFNAR1, IFNAR2, and IL-28R were evaluated by RT-qPCR. Data are from a representative experiment performed in triplicate. *, P<0.05.

FIG. 13B is an image of a Western blot showing the expression of the indicated proteins in A549 cells treated according to the indicated conditions. A549 cells were transfected with the control siRNA, IFN-α/βR or IL-28R siRNA, or a combination of both. After 48 h, cells were treated with 5′ppp-SEQ ID NO: 1 (10 ng/ml) or IFN-a2b (100 UI/ml) for 24 h. Expression of IFIT1, RIG-I, and β-actin was evaluated by Western blotting. The evaluation of 5′ppp-SEQ ID NO: 1-induced immune response by Western blotting in the absence of type I IFN receptor, representative of three independent experiments, and in the absence of type III IFN receptor, representative of one experiment.

FIG. 13C is a bar graph indicating the expression of DENV E protein in A549 cells treated according to the indicated conditions. After siRNA knockdown of IFN-α/βR as described for in FIG. 13B, cells were treated with increasing concentrations of 5′ppp-SEQ ID NO: 1 (0.1 to 10 ng/ml) and then infected with DENV (MOI, 0.1). The percentage of DENV-infected cells was evaluated by flow cytometry. Data are the means±SEM of a representative experiment performed in triplicate. *, P<0.05.

FIG. 13D is an image of a Western Blot showing the expression of the indicated proteins in A549 cells treated according to the indicated conditions. A549 cells were transfected with control and STAT1 siRNA, and 48 h later they were treated with 5′ppp-SEQ ID NO: 1 (0.01 to 1 ng/ml) for 24 h. Expression of STAT1, IFIT1, and β-actin was evaluated by Western blotting. The induction of 5′ppp-SEQ ID NO: 1-induced immune response in the absence of STAT is representative of two independent experiments.

FIG. 13E is a bar graph showing the expression of DENV E protein in A549 cells treated according to the indicated conditions. A549 cells were transfected with control or STAT1 siRNA and incubated for 48 h. Cells were treated with increasing concentrations of 5′ppp-SEQ ID NO: 1 (0.1 to 10 ng/ml) and then infected with DENV (MOI, 0.1). The percentage of DENV-infected cells was evaluated by flow cytometry. Data are the means±SEM from a representative experiment performed in triplicate. *, P<0.05.

FIG. 13F is an image of a Western blot showing the expression of the indicated proteins in A549 cells treated according to the indicated conditions. A549 cells were transfected with control, IRF1, IRF3, or IRF7 siRNA for 48 h, and the protein expression level of these transcription factors was evaluated by Western blotting. This panel is representative of one experiment.

FIG. 13G is a bar graph showing the expression of DENV E protein in A549 cells treated according to the indicated conditions. A549 cells were transfected with control IRF1, IRF3, or IRF7 and then treated as described for panel E. The percentage of DENV-infected cells was evaluated by flow cytometry. Data are the means±SEM from a representative experiment performed in triplicate. *, P<0.05.

FIG. 14A is a set of eight flow cytometry histograms showing the expression of DENV E protein in A549 cells treated according to the indicated conditions. Negatively selected monocytes were challenged with DENV (MOI, 20) in the presence or absence of the enhancing antibody 4G2 (0.5 μg/ml) for 4 h. They were subsequently transfected with 5′ppp-SEQ ID NO: 1 (100 ng/ml) using Lyovec and incubated for 20 h. An IgG2a antibody (0.5 μg/ml) served as a negative control. The percentage of DENV-infected cells was determined by flow cytometry 24 h after infection.

FIG. 14B is a bar graph showing the expression of DENV E protein in A549 cells treated according to the indicated conditions. CD14⁻ MDDCs were challenged with DENV (MOI, 10) for 4 h, followed by transfection with 5′ppp-SEQ ID NO: 1 (100 ng/ml) and incubation for an additional 20 h. Data represent the means±SEM of an experiment performed in triplicate. *, P<0.05.

FIG. 14C is a bar graph showing the percentage of viable A549 cells treated according to the indicated conditions. Cell viability was assessed by flow cytometry on CD14⁻ MDDC and determined 24 h after 5′ppp-SEQ ID NO: 1 treatment (10 to 500 ng/ml) in the presence of Lyovec. Data are the means±SEM of a representative experiment performed in triplicate.

FIG. 14D is an image of a Western blot showing the expression of the indicated proteins in A549 cells treated according to the indicated conditions. CD14⁻ MDDCs were challenged with DENV (MOI, 10) for 4 h and then were treated with 5′ppp-SEQ ID NO: 1 (100 ng/ml) for an additional 20 h. WCEs were resolved by SDS-PAGE and analyzed by immunoblotting for phospho-IRF3, IRF3, phospho-STAT1, STAT1, IFIT1, RIG-I, STING, and β-actin. Results are from one representative experiment that was repeated once.

FIG. 15A is a plot showing reporter gene expression in MRC-5 cells infected with CHIKV LS3-GFP and treated according to the indicated conditions. MRC-5 cells were treated with 0.015 to 4 ng/ml of control RNA or 5′ppp-SEQ ID NO: 1 from 1 h prior to infection to 24 h postinfection with CHIKV LS3-GFP (MOI, 0.1). At 24 h p.i., cells were fixed and EGFP reporter gene expression was quantified. *, P<0.05. cntrl, control.

FIG. 15B is a plot showing cell viability in MRC-5 cells infected with CHIKV LS3-GFP and treated according to the indicated conditions. To assess potential cytotoxicity, MRC-5 cell viability was measured 24 h posttransfection of 5′ppp-SEQ ID NO: 1 or control RNA lacking the 5′ triphosphate. Data are represented as the means±SEM from a representative experiment performed in quadruplicate.

FIG. 15C is an image of a Northern blot showing the intracellular accumulation of CHIKV positive and negative strand RNA in MRC-5 cells treated according to the indicated conditions. The intracellular accumulation of CHIKV positive- and negative-strand RNA was determined by in-gel hybridization of RNA isolated from MRC-5 cells that were treated with 5′ppp-SEQ ID NO: 1 (0.1 to 10 ng/ml) 1 h prior to infection (MOI, 0.1).

FIG. 15D is an image of a Western blot showing the expression of the indicated CHIKV proteins in MRC-5 cells infected with CHIKV and treated according to the indicated conditions. CHIKV E2, E3E2, and nsP1 protein expression was assessed by Western blotting of lysates of MRC-5 cells that were treated with various concentrations of control RNA or 5′ppp-SEQ ID NO: 11 h prior to infection with CHIKV. Data are representative of at least two independent experiments.

FIG. 15E is a bar graph showing the CHIKV titer in MRC-5 cells infected with CHIKV and treated according to the indicated conditions as assessed by plaque assay.

FIG. 15F is a bar graph of reporter gene expression in MRC-5 cells infected with CHIKV LS3-GFP, transfected with the indicated siRNA and treated according to the indicated conditions. siRNA transfected MRC-5 cells were either left untreated or were transfected with 5′ppp-SEQ ID NO: 1, after which they were infected with CHIKV LS3-GFP (MOI, 0.1). CHIKV-driven EGFP reporter gene expression was measured at 24 h p.i. and was normalized to the expression level in CHIKV-infected cells that had been transfected with a nontargeting scrambled siRNA (scr). *, P<0.05.

FIG. 15G is a set of three images of Western blots showing the expression of the indicated proteins in MRC-5 cells infected with CHIKV and treated according to the indicated conditions. MRC-5 cells were transfected with 10 pmol of scrambled siRNA (siScr) or siRNA targeting RIG-I, STAT1, or STING 48 h prior to treatment with 1 ng/ml of 5′ppp-SEQ ID NO: 1. Expression levels of RIG-I, STAT1, STING, and IFIT1 were monitored by Western blotting. Cyclophilin A or B was used as a loading control. Data are representative of at least two independent experiments.

For all of FIGS. 16A, 16B, and 16C, MRC-5 cells were infected with CHIKV LS3-GFP at an MOI of 0.1, and at the indicated time points postinfection they were transfected with 1 ng/ml 5′ ppp-SEQ ID NO: 1, or control RNA.

FIG. 16A is a bar graph of reporter gene expression in MRC-5 cells described above treated according to the indicated conditions. Cells were fixed at 24 h p.i., and EGFP reporter gene expression was quantified and normalized to that in untreated cells. *, P<0.05.

FIG. 16B is a bar graph of CHIKV virus titer in the MRC-5 cells described above. CHIKV progeny titers 24 h p.i. and after 5′ppp-SEQ ID NO: 1 or control RNA treatment were determined by plaque assay.

FIG. 16C is a set of 24 images from Western blots from the cells described above showing the expression of the indicated proteins in cells treated according to the indicated conditions. MRC-5 cells were transfected with 0.1, 1, or 10 ng/ml 5′ppp-SEQ ID NO: 1 or control RNA 1 h prior to infection with CHIKV LS3-GFP (MOI, 0.1). At 24 h p.i., cell lysates were prepared and STAT1, RIG-I, and IFIT-I protein levels were determined by Western blotting. Actin or the transferrin receptor were used as loading controls. Data are representative of at least two independent experiments.

### SEQUENCE LISTING

SEQ ID NO: 1 is an oligoribonucleotide derived from the 5′ UTR and 3′ UTR of vesicular stomatitis virus (VSV).

SEQ ID NO: 2 is the sequence of DNA template encoding the oligoribonucleotide of SEQ ID NO: 1.

SEQ ID NO: 3 is a forward primer for the detection of IFNB1 expression by RT-PCR.

SEQ ID NO: 4 is a reverse primer for the detection of IFNB1 expression by RT-PCR.

SEQ ID NO: 5 is a forward primer for the detection of IL29 expression by RT-PCR.

SEQ ID NO: 6 is a reverse primer for the detection of IL29 expression by RT-PCR.

SEQ ID NO: 7 is a forward primer for the detection of IRF7 expression by RT-PCR.

SEQ ID NO: 8 is a reverse primer for the detection of IRF7 expression by RT-PCR.

SEQ ID NO: 9 is a forward primer for the detection of CCL5 expression by RT-PCR.

SEQ ID NO: 10 is a reverse primer for the detection of CCL5 expression by RT-PCR.

SEQ ID NO: 11 is a forward primer for the detection of CXCL10 expression by RT-PCR.

SEQ ID NO: 12 is a reverse primer for the detection of CXCL10 expression by RT-PCR.

SEQ ID NO: 13 is a forward primer for the detection of ILE expression by RT-PCR.

SEQ ID NO: 14 is a reverse primer for the detection of ILE expression by RT-PCR.

SEQ ID NO: 15 is a forward primer for the detection of ISG15 expression by RT-PCR.

SEQ ID NO: 16 is a reverse primer for the detection of ISG15 expression by RT-PCR.

SEQ ID NO: 17 is a forward primer for the detection of ISG56 expression by RT-PCR.

SEQ ID NO: 18 is a reverse primer for the detection of ISG56 expression by RT-PCR.

SEQ ID NO: 19 is a forward primer for the detection of RIG-I expression by RT-PCR.

SEQ ID NO: 20 is a reverse primer for the detection of RIG-I expression by RT-PCR.

SEQ ID NO: 21 is a forward primer for the detection of Viperine expression by RT-PCR.

SEQ ID NO: 22 is a reverse primer for the detection of Viperine expression by RT-PCR.

SEQ ID NO: 23 is a forward primer for the detection of OASL expression by RT-PCR.

SEQ ID NO: 24 is a reverse primer for the detection of OASL expression by RT-PCR.

SEQ ID NO: 25 is a forward primer for the detection of NOXA expression by RT-PCR.

SEQ ID NO: 26 is a reverse primer for the detection of NOXA expression by RT-PCR.

SEQ ID NO: 27 is a forward primer for the detection of GADPH expression by RT-PCR.

SEQ ID NO: 28 is a reverse primer for the detection of GADPH expression by RT-PCR.

SEQ ID NO: 29 is a forward primer for the detection of Dengue virus RNA expression by RT-PCR.

SEQ ID NO: 30 is a reverse primer for the detection of Dengue virus RNA expression by RT-PCR.

SEQ ID NO: 31 is a forward primer for the detection of DENV2

SEQ ID NO: 32 is a reverse primer for the detection of DENV2.

SEQ ID NO: 33 is a forward primer for the detection of GADPH.

SEQ ID NO: 34 is a reverse primer for the detection of GADPH.

SEQ ID NO: 35 is a forward primer for the detection of IFNα2.

SEQ ID NO: 36 is a reverse primer for the detection of IFNα2.

SEQ ID NO: 37 is a forward primer for the detection of IFNAR1.

SEQ ID NO: 38 is a reverse primer for the detection of IFNAR1.

SEQ ID NO: 39 is a forward primer for the detection of IFNAR2.

SEQ ID NO: 40 is a reverse primer for the detection of IFNAR2.

SEQ ID NO: 41 is a forward primer for the detection of IFNB1

SEQ ID NO: 42 is a reverse primer for the detection of IFNB1

SEQ ID NO: 43 is a forward primer for the detection of ILA.

SEQ ID NO: 44 is a reverse primer for the detection of ILA.

SEQ ID NO: 45 is a forward primer for the detection of IL-6.

SEQ ID NO: 46 is a reverse primer for the detection of IL-6.

SEQ ID NO: 47 is a forward primer for the detection of IL28RA.

SEQ ID NO: 48 is a reverse primer for the detection of IL28RA.

SEQ ID NO: 49 is a forward primer for the detection of IL-29.

SEQ ID NO: 50 is a reverse primer for the detection of IL-29.

SEQ ID NO: 51 is a forward primer for the detection of TNFα

SEQ ID NO: 52 is a reverse primer for the detection of TNFα.

SEQ ID NO: 53 is the CHIKVhyb4 probe.

SEQ ID NO: 54 is the CHIKVhyb2 probe.

### DETAILED DESCRIPTION

Disclosed herein is a oligoribonucleotide of SEQ ID NO: 1 comprising a triphosphate group on the 5′ end (5′ppp-SEQ ID NO: 1), pharmaceutical compositions comprising the oligoribonucleotide, and methods of using the oligoribonucleotide to treat viral infections.

A DNA plasmid may be used to generate an oligoribonucleotide of SEQ ID NO: 1. Such a plasmid may include SEQ ID NO: 2. The oligoribonucleotide can be transcribed as an RNA molecule that automatically folds into duplexes with hairpin loops. Typically, a transcriptional unit or cassette will contain an RNA transcript promoter sequence, such as a T7 promoter operably linked to SEQ ID NO: 2 for transcription of 5′ppp-SEQ ID NO: 1.

Methods of isolating RNA, synthesizing RNA, hybridizing nucleic acids, making and screening cDNA libraries, and performing PCR are well known in the art (see, e.g., Gubler and Hoffman, Gene 25, 263-269 (1983); Sambrook and Russell, Molecular Cloning: A Laboratory Manual, Cold Spring Harbor Laboratory Press, Cold Spring Harbor N.Y., (2001)) as are PCR methods (see, U.S. Pat. Nos. 4,683,195 and 4,683,202; PCR Protocols: A Guide to Methods and Applications, Innis et al, eds, (1990)). Expression libraries are also well known to those of skill in the art. Additional basic texts disclosing the general methods of use in this invention include Sambrook and Russell (2001) supra; Kriegler, Gene Transfer and Expression: A Laboratory Manual (1990); and Current Protocols in Molecular Biology (Ausubel et al., eds., 1994).

An oligoribonucleotide may be chemically synthesized. Synthesis of the single-stranded nucleic acid makes use of common nucleic acid protecting and coupling groups, such as dimethoxytrityl at the 5′-end and phosphoramidites at the 3′-end. As a non-limiting example, small scale syntheses can be conducted on an Applied Biosystems synthesizer using a 0.2 micromolar scale protocol with a 2.5 min coupling step for 2′-O-methylated nucleotides. Alternatively, syntheses at the 0.2 micromolar scale can be performed on a 96-well plate synthesizer from Protogene. However, a larger or smaller scale of synthesis is encompassed by the invention, including any method of synthesis now known or yet to be disclosed. Suitable reagents for synthesis of the siRNA single-stranded molecules, methods for RNA deprotection, and methods for RNA purification are known to those of skill in the art.

An oligoribonucleotide can be synthesized via a tandem synthesis technique, wherein both strands are synthesized as a single continuous fragment or strand separated by a linker that is subsequently cleaved to provide separate fragments or strands that hybridize to form an RNA duplex. The linker may be any linker, including a polynucleotide linker or a non-nucleotide linker. The tandem synthesis of RNA can be readily adapted to both multiwell/multiplate synthesis platforms as well as large scale synthesis platforms employing batch reactors, synthesis columns, and the like. Alternatively, the oligoribonucleotide can be assembled from two distinct single-stranded molecules, wherein one strand includes the sense strand and the other includes the antisense strand of the RNA. For example, each strand can be synthesized separately and joined together by hybridization or ligation following synthesis and/or deprotection. Either the sense or the antisense strand may contain additional nucleotides that are not complementary to one another and do not form a double stranded RNA molecule. In certain other instances, the oligoribonucleotide can be synthesized as a single continuous fragment, where the self-complementary sense and antisense regions hybridize to form an RNA duplex having a hairpin or panhandle secondary structure.

An oligoribonucleotide may comprise a duplex having two complementary strands that form a double-stranded region with least one modified nucleotide in the double-stranded region. The modified nucleotide may be on one strand or both. If the modified nucleotide is present on both strands, it may be in the same or different positions on each strand. Examples of modified nucleotides suitable for use in the present invention include, but are not limited to, ribonucleotides having a 2′-O-methyl (2′OMe), 2′-deoxy-2′-fluoro (2′F), 2′-deoxy, 5-C-methyl, 2′-O-(2-methoxyethyl) (MOE), 4′-thio, 2′-amino, or 2′-C-allyl group. Modified nucleotides having a conformation such as those described in, for example in Sanger, Principles of Nucleic Acid Structure, Springer-Verlag Ed. (1984), are also suitable for use in oligoribonucleotides. Other modified nucleotides include, without limitation: locked nucleic acid (LNA) nucleotides, G-clamp nucleotides, or nucleotide base analogs. LNA nucleotides include but need not be limited to 2′-0,4′-C-methylene-(D-ribofuranosyl)nucleotides), 2′-O-(2-methoxyethyl) (MOE) nucleotides, 2′-methyl-thio-ethyl nucleotides, 2′-deoxy-2′-fluoro (2′F) nucleotides, 2′-deoxy-2′-chloro (2Cl) nucleotides, and 2′-azido nucleotides. A G-clamp nucleotide refers to a modified cytosine analog wherein the modifications confer the ability to hydrogen bond both Watson-Crick and Hoogsteen faces of a complementary guanine nucleotide within a duplex (Lin et al, J Am Chem Soc, 120, 8531-8532 (1998)). Nucleotide base analogs include for example, C-phenyl, C-naphthyl, other aromatic derivatives, inosine, azole carboxamides, and nitroazole derivatives such as 3-nitropyrrole, 4-nitroindole, 5-nitroindole, and 6-nitroindole (Loakes, Nucl Acids Res, 29, 2437-2447 (2001)).

An oligoribonucleotide may comprise one or more chemical modifications such as terminal cap moieties, phosphate backbone modifications, and the like. Examples of classes of terminal cap moieties include, without limitation, inverted deoxy abasic residues, glyceryl modifications, 4′,5′-methylene nucleotides, 1-(β-D-erythrofuranosyl) nucleotides, 4′-thio nucleotides, carbocyclic nucleotides, 1,5-anhydrohexitol nucleotides, L-nucleotides, α-nucleotides, modified base nucleotides, threo pentofuranosyl nucleotides, acyclic 3′,4′-seco nucleotides, acyclic 3,4-dihydroxybutyl nucleotides, acyclic 3,5-dihydroxypentyl nucleotides, 3′-3′-inverted nucleotide moieties, 3′-3′-inverted abasic moieties, 3′-2′-inverted nucleotide moieties, 3′-2′-inverted abasic moieties, 5′-5′-inverted nucleotide moieties, 5′-5′-inverted abasic moieties, 3′-5′-inverted deoxy abasic moieties, 5′-amino-alkyl phosphate, 1,3-diamino-2-propyl phosphate, 3 aminopropyl phosphate, 6-aminohexyl phosphate, 1,2-aminododecyl phosphate, hydroxypropyl phosphate, 1,4-butanediol phosphate, 3′-phosphoramidate, 5′ phosphoramidate, hexylphosphate, aminohexyl phosphate, 3′-phosphate, 5′-amino, 3′-phosphorothioate, 5′-phosphorothioate, phosphorodithioate, and bridging or non-bridging methylphosphonate or 5′-mercapto moieties (see, e.g., U.S. Pat. No. 5,998,203; Beaucage et al, Tetrahedron 49, 1925 (1993)). Non-limiting examples of phosphate backbone modifications (i.e., resulting in modified internucleotide linkages) include phosphorothioate, phosphorodithioate, methylphosphonate, phosphotriester, morpholino, amidate, carbamate, carboxymethyl, acetamidate, polyamide, sulfonate, sulfonamide, sulfamate, formacetal, thioformacetal, and alkylsilyl substitutions (see, e.g., Hunziker et al, Modern Synthetic Methods, VCH, 331-417 (1995); Mesmaeker et al, Antisense Research, ACS, 24-39 (1994)). Such chemical modifications can occur at the 5′-end and/or 3′-end of the sense strand, antisense strand, or both strands of the oligoribonucleotide.

The sense and/or antisense strand of an oligoribonucleotide may comprise a 3′-terminal overhang having 1 to 4 or more 2′-deoxyribonucleotides and/or any combination of modified and unmodified nucleotides. Additional examples of modified nucleotides and types of chemical modifications that can be introduced into the modified oligoribonucleotides of the present invention are described, e.g., in UK Patent No. GB 2,397,818 B and U.S. Patent Publication Nos. 20040192626 and 20050282188.

An oligoribonucleotide may comprise one or more non-nucleotides in one or both strands of the siRNA. A non-nucleotide may be any subunit, functional group, or other molecular entity capable of being incorporated into a nucleic acid chain in the place of one or more nucleotide units that is not or does not comprise a commonly recognized nucleotide base such as adenosine, guanine, cytosine, uracil, or thymine, such as a sugar or phosphate.

Chemical modification of the oligoribonucleotide may also comprise attaching a conjugate to the oligoribonucleotide molecule. The conjugate can be attached at the 5′- and/or the 3′-end of the sense and/or the antisense strand of the oligoribonucleotide via a covalent attachment such as a nucleic acid or non-nucleic acid linker. The conjugate can also be attached to the oligoribonucleotide through a carbamate group or other linking group (see, e.g., U.S. Patent Publication Nos. 20050074771, 20050043219, and 20050158727). A conjugate may be added to the oligoribonucleotide for any of a number of purposes. For example, the conjugate may be a molecular entity that facilitates the delivery of the oligoribonucleotide into a cell or the conjugate a molecule that comprises a drug or label.

Examples of conjugate molecules suitable for attachment to the disclosed oligoribonucleotides include, without limitation, steroids such as cholesterol, glycols such as polyethylene glycol (PEG), human serum albumin (HSA), fatty acids, carotenoids, terpenes, bile acids, folates (e.g., folic acid, folate analogs and derivatives thereof), sugars (e.g., galactose, galactosamine, N-acetyl galactosamine, glucose, mannose, fructose, fucose, etc.), phospholipids, peptides, ligands for cellular receptors capable of mediating cellular uptake, and combinations thereof (see, e.g., U.S. Patent Publication Nos. 20030130186, 20040110296, and 20040249178; U.S. Pat. No. 6,753,423). Other examples include the lipophilic moiety, vitamin, polymer, peptide, protein, nucleic acid, small molecule, oligosaccharide, carbohydrate cluster, intercalator, minor groove binder, cleaving agent, and cross-linking agent conjugate molecules described in U.S. Patent Publication Nos. 20050119470 and 20050107325. Other examples include the 2′-O-alkyl amine, 2′-O-alkoxyalkyl amine, polyamine, C5-cationic modified pyrimidine, cationic peptide, guanidinium group, amidininium group, cationic amino acid conjugate molecules described in U.S. Patent Publication No. 20050153337. Additional examples of conjugate molecules include a hydrophobic group, a membrane active compound, a cell penetrating compound, a cell targeting signal, an interaction modifier, or a steric stabilizer as described in U.S. Patent Publication No. 20040167090. Further examples include the conjugate molecules described in U.S. Patent Publication No. 20050239739.

The type of conjugate used and the extent of conjugation to the oligoribonucleotide can be evaluated for improved pharmacokinetic profiles, bioavailability, and/or stability of the oligoribonucleotide while retaining activity. As such, one skilled in the art can screen oligoribonucleotides having various conjugates attached thereto to identify oligonucleotide conjugates having improved properties using any of a variety of well-known in vitro cell culture or in vivo animal models.

An oligoribonucleotide may be incorporated into a pharmaceutically acceptable carrier or transfection reagent containing the oligoribonucleotides described herein. The carrier system may be a lipid-based carrier system such as a stabilized nucleic acid-lipid particle (e.g., SNALP or SPLP), cationic lipid or liposome nucleic acid complexes (i.e., lipoplexes), a liposome, a micelle, a virosome, or a mixture thereof. In other embodiments, the carrier system is a polymer-based carrier system such as a cationic polymer-nucleic acid complex (i.e., polyplex). In additional embodiments, the carrier system is a cyclodextrin-based carrier system such as a cyclodextrin polymer-nucleic acid complex (see US Patent Application Publication 20070218122). In further embodiments, the carrier system is a protein-based carrier system such as a cationic peptide-nucleic acid complex. An oligoribonucleotide molecule may also be delivered as naked RNA.

A pharmaceutical composition may be any chemical compound or composition capable of inducing a desired therapeutic or prophylactic effect when properly administered to a subject. A pharmaceutical composition can include a therapeutic agent, a diagnostic agent or a pharmaceutical agent. A therapeutic or pharmaceutical agent is one that alone or together with an additional compound induces the desired response (such as inducing a therapeutic or prophylactic effect when administered to a subject). In a particular example, a pharmaceutical agent is an agent that significantly reduces one or more symptoms associated with viral infection. A pharmaceutical composition may be a member of a group of compounds. Pharmaceutical compositions may be grouped by any characteristic including chemical structure and the molecular target they affect.

A pharmaceutically acceptable carrier (interchangeably termed a vehicle) may be any material or molecular entity that facilitates the administration or other delivery of the pharmaceutical composition. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle.

A therapeutically effective amount or concentration of a compound such as 5′ppp-SEQ ID NO: 1 may be any amount of a composition that alone, or together with one or more additional therapeutic agents is sufficient to achieve a desired effect in a subject, or in a cell being treated with the agent. The effective amount of the agent will be dependent on several factors, including, but not limited to, the subject or cells being treated and the manner of administration of the therapeutic composition. In one example, a therapeutically effective amount or concentration is one that is sufficient to prevent advancement, delay progression, or to cause regression of a disease, or which is capable of reducing symptoms caused by any disease, including viral infection.

In one example, a desired effect is to reduce or inhibit one or more symptoms associated with viral infection. The one or more symptoms do not have to be completely eliminated for the composition to be effective. For example, a composition can decrease the sign or symptom by a desired amount, for example by at least 20%, at least 50%, at least 80%, at least 90%, at least 95%, at least 98%, or even at least 100%, as compared to the sign or symptom in the absence of the composition.

A therapeutically effective amount of a pharmaceutical composition can be administered in a single dose, or in several doses, for example daily, during a course of treatment. However, the therapeutically effective amount can depend on the subject being treated, the severity and type of the condition being treated, and the manner of administration. For example, a therapeutically effective amount of such agent can vary from about 100 μg-10 mg per kg body weight if administered intravenously.

The actual dosages will vary according to factors such as the type of virus to be protected against and the particular status of the subject (for example, the subject's age, size, fitness, extent of symptoms, susceptibility factors, and the like) time and route of administration, other drugs or treatments being administered concurrently, as well as the specific pharmacology of treatments for viral infection for eliciting the desired activity or biological response in the subject. Dosage regimens can be adjusted to provide an optimum prophylactic or therapeutic response.

A therapeutically effective amount is also one in which any toxic or detrimental side effects of the compound and/or other biologically active agent is outweighed in clinical terms by therapeutically beneficial effects. A non-limiting range for a therapeutically effective amount of treatments for viral infection within the methods and formulations of the disclosure is about 0.0001 μg/kg body weight to about 10 mg/kg body weight per dose, such as about 0.0001 μg/kg body weight to about 0.001 μg/kg body weight per dose, about 0.001 μg/kg body weight to about 0.01 μg/kg body weight per dose, about 0.01 μg/kg body weight to about 0.1 μg/kg body weight per dose, about 0.1 μg/kg body weight to about 10 μg/kg body weight per dose, about 1 μg/kg body weight to about 100 μg/kg body weight per dose, about 100 μg/kg body weight to about 500 μg/kg body weight per dose, about 500 μg/kg body weight per dose to about 1000 μg/kg body weight per dose, or about 1.0 mg/kg body weight to about 10 mg/kg body weight per dose.

Dosage can be varied by the attending clinician to maintain a desired concentration. Higher or lower concentrations can be selected based on the mode of delivery, for example, trans-epidermal, rectal, oral, pulmonary, intranasal delivery, intravenous or subcutaneous delivery.

Determination of effective amount is typically based on animal model studies followed up by human clinical trials and is guided by administration protocols that significantly reduce the occurrence or severity of targeted disease symptoms or conditions in the subject. Suitable models in this regard include, for example, murine, rat, porcine, feline, non-human primate, and other accepted animal model subjects known in the art. Alternatively, effective dosages can be determined using in vitro models (for example, viral titer assays or cell culture infection assays). Using such models, only ordinary calculations and adjustments are required to determine an appropriate concentration and dose to administer a therapeutically effective amount of the treatments for viral infection (for example, amounts that are effective to alleviate one or more symptoms of viral infection).

### Methods of Treating Viral Infections

Disclosed herein are methods of treating a subject that has or may have a viral infection comprising administering a pharmaceutical composition comprising 5′ppp-SEQ ID NO: 1 to the subject. The subject may be treated therapeutically or prophylactically.

A subject may be any multi-cellular vertebrate organisms, a category that includes human and non-human mammals, such as mice. In some examples a subject is a male. In some examples a subject is a female. Further types of subjects to which the pharmaceutical composition may be properly administered include subjects known to have a viral infection (through, for example, a molecular diagnostic test or clinical diagnosis,) subjects having a predisposition to contracting a viral infection (for example by living in or travelling to a region in which one or more viruses is endemic), or subjects displaying one or more symptoms of having a viral infection.

Administration of a pharmaceutical composition may be any method of providing or give a subject a pharmaceutical composition comprising 5′ppp-SEQ ID NO: 1, by any effective route. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes.

Treating a subject may be any therapeutic intervention that ameliorates a sign or symptom of a disease or pathological condition after it has begun to develop, whether or not the subject has developed symptoms of the disease. Ameliorating, with reference to a disease, pathological condition or symptom refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the memory and/or cognitive function of the subject, a qualitative improvement in symptoms observed by a clinician or reported by a patient, or by other parameters well known in the art that are specific to viral infections generally or specific viral infections.

A symptom may be any subjective evidence of disease or of a subject's condition, for example, such evidence as perceived by the subject; a noticeable change in a subject's condition indicative of some bodily or mental state. A sign may be any abnormality indicative of disease, discoverable on examination or assessment of a subject. A sign is generally an objective indication of disease.

The administration of a pharmaceutical composition comprising 5′ppp-SEQ ID NO: 1 can be for either prophylactic or therapeutic purposes. When provided prophylactically, the treatments are provided in advance of any clinical symptom of viral infection. Prophylactic administration serves to prevent or ameliorate any subsequent disease process. When provided therapeutically, the compounds are provided at (or shortly after) the onset of a symptom of disease. For prophylactic and therapeutic purposes, the treatments can be administered to the subject in a single bolus delivery, via continuous delivery (for example, continuous transdermal, mucosal or intravenous delivery) over an extended time period, or in a repeated administration protocol (for example, by an hourly, daily or weekly, repeated administration protocol). The therapeutically effective dosage of the treatments for viral infection can be provided as repeated doses within a prolonged prophylaxis or treatment regimen that will yield clinically significant results to alleviate one or more symptoms or detectable conditions associated with viral infection.

Suitable methods, materials, and examples used in the practice and/or testing of embodiments of the disclosed invention are described below. Such methods and materials are illustrative only and are not intended to be limiting. Other methods, materials, and examples similar or equivalent to those described herein can be used.

### EXAMPLES

The following examples are illustrative of disclosed methods. In light of this disclosure, those of skill in the art will recognize that variations of these examples and other examples of the disclosed method would be possible without undue experimentation.

### Example 1

### 5′-Ppp-SEQ ID NO: 1 Stimulates an Antiviral Response in Lung Epithelial A549 Cells

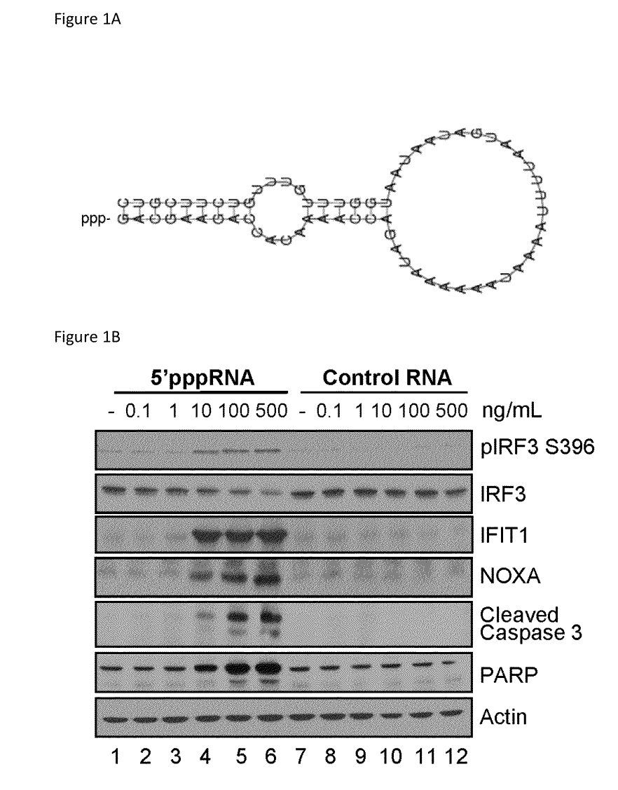

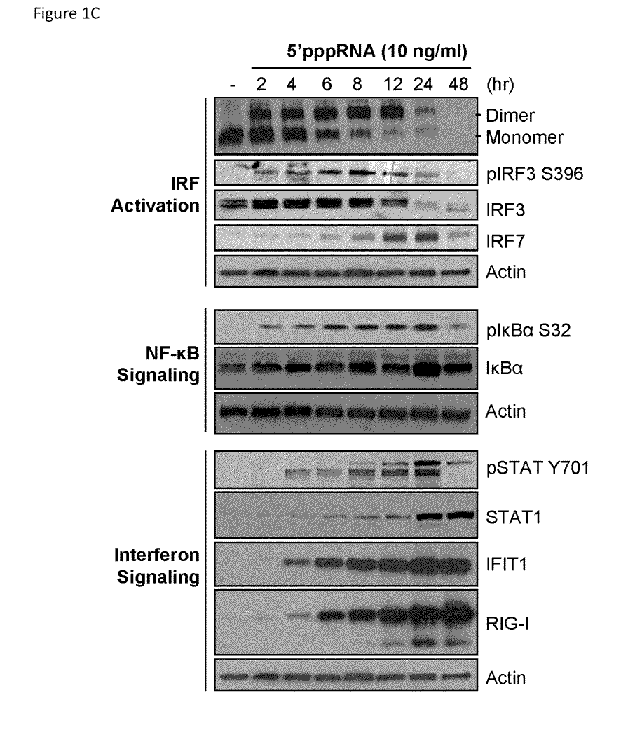

A short RNA oligomer derived from the 5′ and 3′ UTRs of the negative-strand RNA virus Vesicular Stomatitis Virus (VSV) was generated by in vitro transcription using T7 polymerase, an enzymatic reaction that synthesizes RNA molecules with a 5′ ppp terminus (5′-ppp-SEQ ID NO: 1). The predicted panhandle secondary structure of the 5′ppp-SEQ ID NO: 1 is depicted in FIG. 1A. Gel analysis and nuclease sensitivity confirmed the synthesis of a single RNA product of the expected length of 67 nucleotides.

The transfection of 5′ppp-SEQ ID NO: 1 into A549 cells resulted in Ser396 phosphorylation of IRF3 at 8 hours—a hallmark of immediate early activation of the antiviral response (FIG. 1B, see particularly lanes 2 to 6). Induction of apoptosis was also detected following treatment with higher concentrations of 5′-ppp-SEQ ID NO: 1. Furthermore, the pro-apoptotic protein NOXA—a direct transcriptional target of IRF3—as well as cleavage products of caspase 3 and PARP were up-regulated in a dose dependent manner upon transfection with 5′ppp-SEQ ID NO: 1.) (See Gobau D et al, Eur J Immunol 39, 527-540 (2009), incorporated by reference herein). Optimal induction of antiviral signaling with limited cytotoxicity was achieved at a concentration of 10 ng/ml (about 500 μM) (FIG. 1B; lane 4). The stimulation of immune signaling and apoptosis was dependent on the 5′ ppp moiety. A homologous RNA without a 5′ ppp terminus did not stimulate immune signaling and apoptosis over a range of RNA concentrations (FIG. 1B, lanes 8 to 12).

To characterize the antiviral response triggered by 5′ppp-SEQ ID NO: 1, the kinetics of downstream RIG-I signaling were measured at different times (0-48 hours) after stimulation of A549 cells (FIG. 1C). IRF3 homodimerization (top panel) and IRF3 phosphorylation at Ser396 (2nd panel) were first detected as early as 2 hours post treatment with 5′ppp-SEQ ID NO: 1 and remained until 24 hours post treatment. Expression of endogenous IRF7 was detected later than that of IRF3 (4th panel vs. 3rd panel). IκBα phosphorylation was detected as early as 2 hours post-treatment and was sustained throughout the time course (6th panel). IRF3, IRF7 and NF-κB are required for optimal induction of the IFNβ promoter.

Tyr701 phosphorylation of STAT1, indicative of JAK-STAT signaling was first detected at 4 hours post treatment with 5′ppp-SEQ ID NO: 1 (9th panel). Tyr 701 phosphorylation was still detected at 24 hours post treatment (10th panel). IFIT1 and RIG-I were both upregulated 4 hours following treatment (11th and 12th panel) while STAT1 and IRF7 (4th and 10th panel) were upregulated 6 hours and 8 hours after treatment (respectively). IFNβ was detectable in cell culture supernatant as early as 6 hours after treatment with a peak concentration of 4000 pg/ml between 12 and 24 hours after treatment (FIG. 1D, top panel). IFNα was first detected at 12 hours after treatment and remained at a concentration of 400 pg/ml throughout the rest of the time course (FIG. 1D, bottom panel).

### Example 2

### 5′-ppp-SEQ ID NO: 1 Induction of the Antiviral Response Requires an Intact RIG-I Pathway

To address whether 5′ppp-SEQ ID NO: 1 exclusively activates RIG-I, wild type mouse embryonic fibroblasts (wtMEF) and RIG-I^{-/-} MEF were co-transfected with 5′ppp-SEQ ID NO: 1 and type 1 IFN reporter constructs to measure promoter activity. 5′ppp-SEQ ID NO: 1 activated the IFNβ promoter 60-fold and the IFNα promoter 450-fold in wtMEF. However, 5′ppp-SEQ ID NO: 1 activated neither promoter in RIG-I^{-/-} MEF.

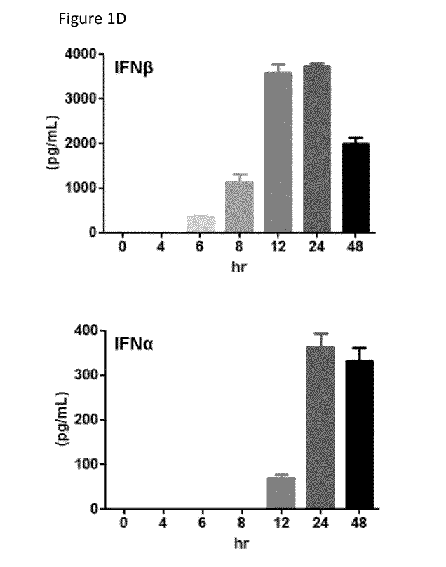

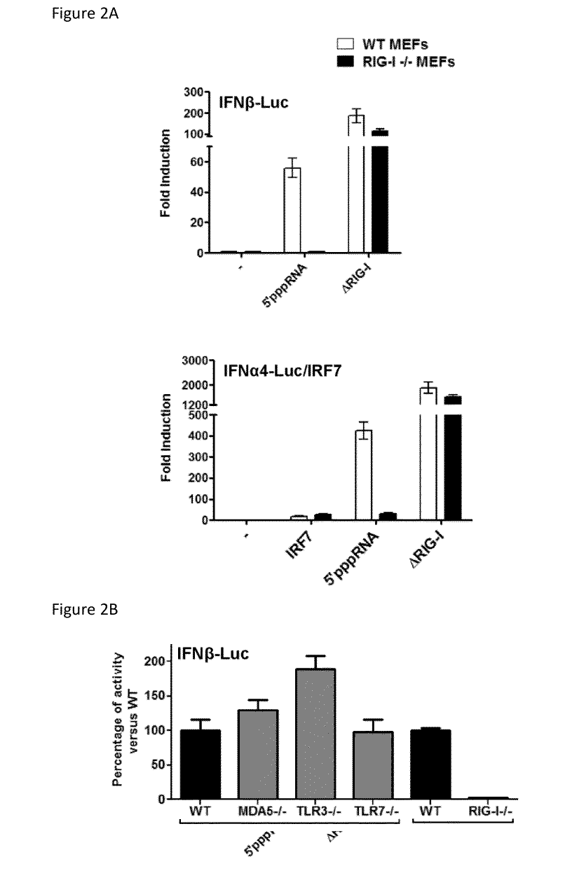

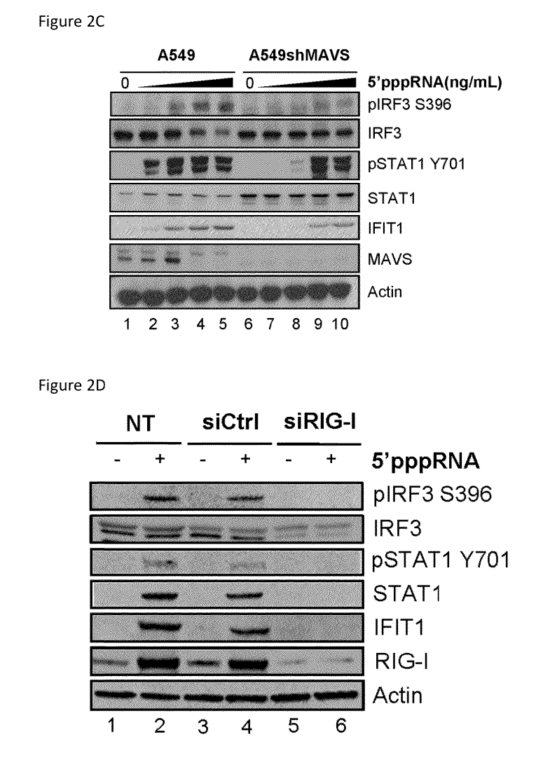

A constitutively active RIG-I mutant (described in Yoneyama M et al, Nat Immunol 5, 730-737 (2004); incorporated by reference herein) was used in a similar experiment (FIG. 2A). Induction of the IFN response by 5′ppp-SEQ ID NO: 1 was dependent on an intact RIG-I signaling pathway because IFNβ promoter activity was unchanged by treatment with 5′ppp-SEQ ID NO: 1 in Mda5^{-/-}, TLR3^{-/-}, or TLR7^{-/-} MEFs (FIG. 2B). In A549 cells treated with 5′ppp-SEQ ID NO: 1, in which RIG-I expression was silenced using siRNA, IRF3 and STAT1 phosphorylation as well as IFIT1 and RIG-I upregulation were inhibited when compared to control cells treated with an irrelevant siRNA. Transient transfection of irrelevant and specific siRNA did not activate immune signaling (FIGS. 2C and 2D).

### Example 3

### 5′-ppp-SEQ ID NO: 1 Acts as a Broad-Spectrum Antiviral Agent

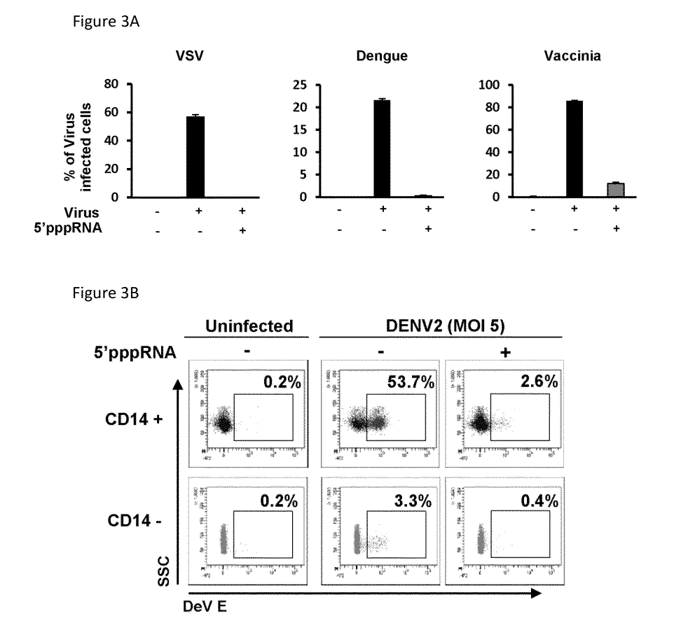

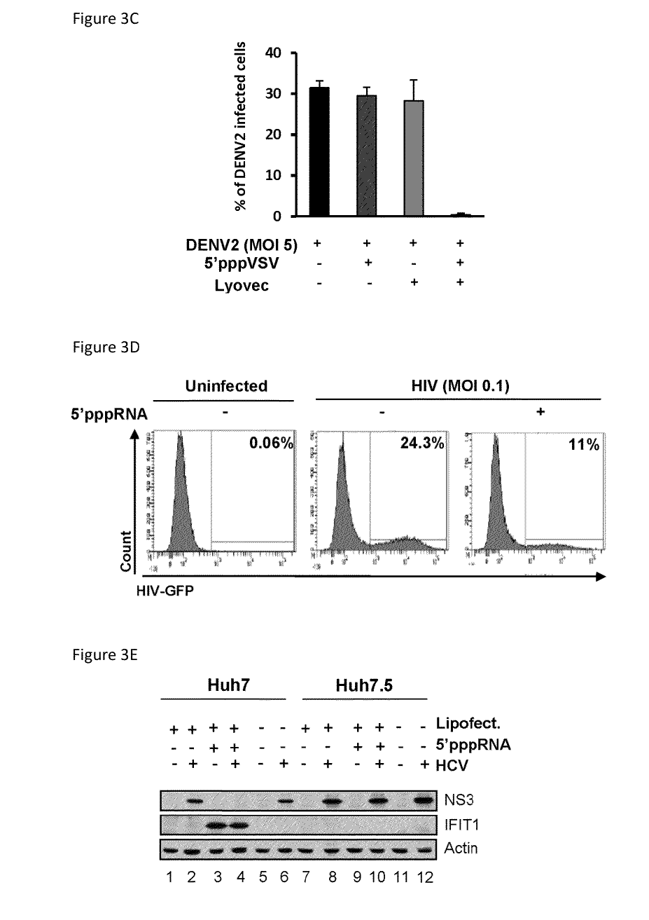

A549 cells were treated with 5′ppp-SEQ ID NO: 1 and 24 hours later were infected with VSV, Dengue (DENV), or Vaccinia viruses. All viruses were able to infect untreated cells (60%, 20% and 80%, respectively as assessed by flow cytometry). In cells pretreated with 5′ppp-SEQ ID NO: 1, VSV and DENV infectivity was less than 0.5%, while infection with vaccinia was about 10% (FIG. 3A). Release of infectious VSV and DENV was blocked by treatment with 5′ppp-SEQ ID NO: 1. VSV infection produced 1.7×10⁹ pfu/ml in untreated cells. No plaque forming units were detectable in cells pretreated with 5′ppp-SEQ ID NO: 1. Similarly, DENV infection produced 4.3×10⁶ pfu/ml in untreated cells while no plaque forming units were detectable in cells pretreated with 5′ppp-SEQ ID NO: 1. In primary human CD14⁺ monocytes, DENV infection was 53.7%, compared to 2.6% infection in CD14⁺ monocytes pretreated with 5′ppp-SEQ ID NO: 1. In CD14⁻ monocytes, DENV infectivity was 3% in untreated cells, but in 0.4% in cells pretreated with 5′ppp-SEQ ID NO: 1 (FIG. 3B).

In another experiment, primary CD14⁺ monocytes from three human subjects were infected with DENV and treated with 5′ppp-SEQ ID NO: 1 alone, transfection reagent alone or 5′ppp-SEQ ID NO: 1 with transfection agent. 5′ppp-SEQ ID NO: 1 alone or transfection agent alone resulted in an infection rate of about 30%, while cells treated with both transfection agent and 5′ppp-SEQ ID NO: 1 had an infection rate of about 0.5% (FIG. 3C).

To evaluate the antiviral effect of 5′ppp-SEQ ID NO: 1 against HIV infection, activated CD4⁺ T cells were pre-treated with supernatant isolated from 5′ppp-SEQ ID NO: 1 treated monocytes and then infected with HIV-GFP (MOI=0.1). In the absence of treatment with the supernatant, 24% of the activated CD4+ T cells were infected by HIV. In cells treated with the supernatant, 11% of the cells were infected (FIG. 3D).

5′ppp-SEQ ID NO: 1 also has an antiviral effect against HCV in the hepatocellular carcinoma cell line Huh7. Expression of HCV NS3 was inhibited by 5′ppp-SEQ ID NO: 1 treatment (FIG. 3E; lane 4 vs. 2 and 6). The antiviral effect was dependent on RIG-I. Huh7.5 cells have a mutant inactive RIG-I. These cells did not upregulate IFIT1 upon 5′ppp-SEQ ID NO: 1 treatment (FIG. 3E; lane 9). Furthermore, NS3 expression Huh7.5 cells was comparable to that of untreated HCV-infected cells (FIG. 3E; lane 10 vs. 8 and 12).

### Example 4

### 5′-Ppp-SEQ ID NO: 1 Inhibits H1N1 Influenza Infection In Vitro

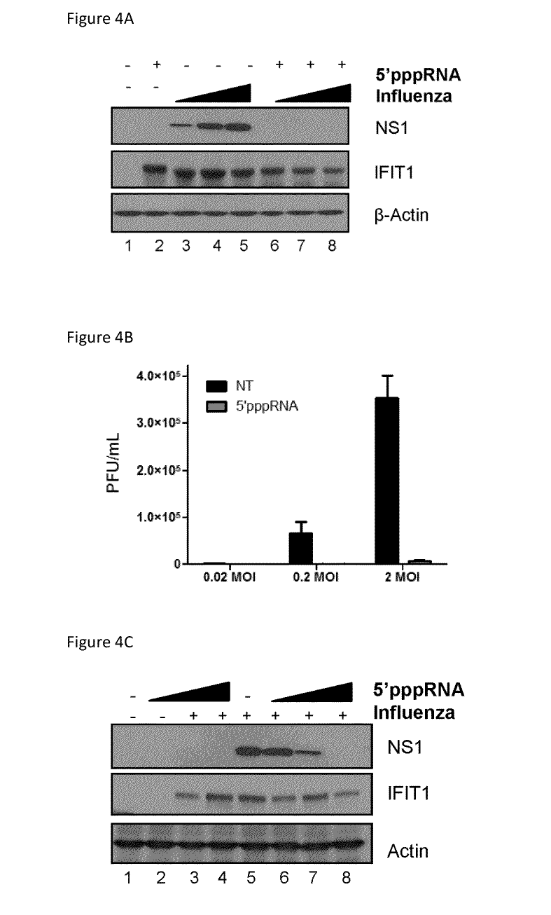

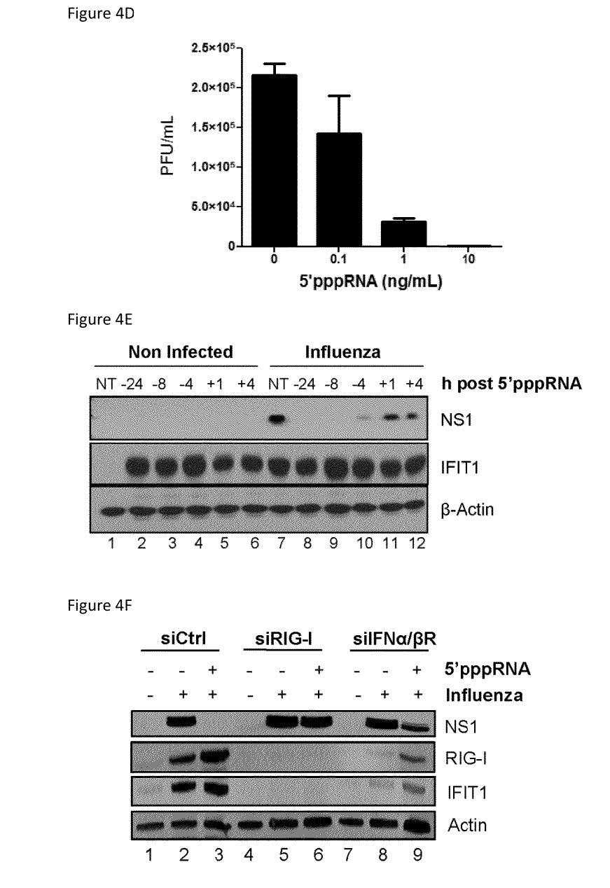

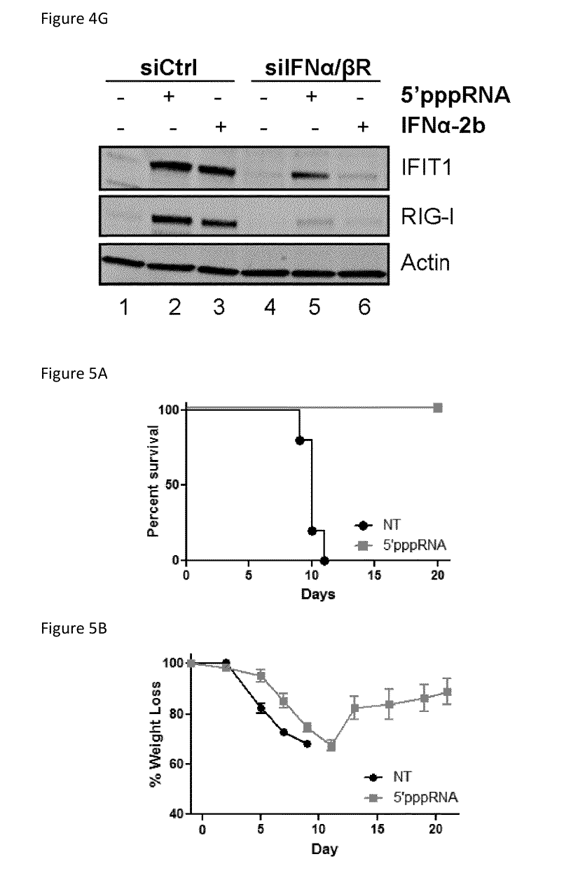

A549 cells were pre-treated with 5′ppp-SEQ ID NO: 1 for 24 hours and then infected with H1N1 A/PR/8/34 Influenza virus at increasing MOI ranging from 0.02 to 2. Influenza replication was monitored by immunoblot analysis of NS1 protein expression (FIG. 4A) and plaque assay (FIG. 4B). Viral replication was blocked by 5′ppp-SEQ ID NO: 1 pre-treatment as demonstrated by a complete loss of NS1 expression and a 40-fold decrease in viral titer at an MOI of 2. In another experiment, A549 cells were pre-treated with decreasing concentrations of 5′ppp-SEQ ID NO: 1 (10 to 0.1 ng/ml) prior to influenza virus challenge at 0.2 MOI. 5′ppp-SEQ ID NO: 1 significantly blocked influenza replication at a concentration of 1 ng/ml with a 3-fold reduction in NS1 protein expression (FIG. 4C; lane 7) and a 7-fold reduction in virus titer by plaque assay (FIG. 4D).

In another experiment, A549 cells were treated with a single dose of 5′ppp-SEQ ID NO: 1 pre- (−24 hours, −8 hours, −4 hours) and post- (+1 hour, +4 hours) influenza challenge. As shown by NS1 expression, pre-treatment with 10 ng/ml 5′ppp-SEQ ID NO: 1 for 8 hours caused a 100-fold reduction in influenza NS1 expression (FIG. 4E, lane 9). Pre-treatment for 4 hours was also effective and resulted in an 8-fold reduction in NS1 (FIG. 4E; lane 10). Additionally, treatment at both 1 and 4 hours post-infection also reduced influenza NS1 expression by 2-fold (FIG. 4E; lanes 11 and 12).

In another experiment siRNA was used to silence RIG-I or IFNα/β receptor in A549 cells that were later infected with influenza. Note that ISG's were not induced by the siRNA (FIG. 4F, lanes 3 vs. 6). 5′ppp-SEQ ID NO: 1 treatment did not inhibit NS1 expression in these infected cells (FIG. 4F; lanes 5 vs. 6). In cells with IFNα/βR expression silenced, there was no IFIT1 or RIG-I expression following treatment with IFNα-2b (FIG. 4G; lane 6). Expression of ISGs was only partially reduced following treatment with 5′ppp-SEQ ID NO: 1. There was a 2.2-fold reduction of IFIT1 in cells with a silenced with IFNα/βR siRNA relative to the negative control siRNA (FIG. 4G; lane 5 vs. 2). However, in those cells, 5′ppp-SEQ ID NO: 1 treatment reduced viral NS1 expression by 2.4-fold (FIG. 4F; lane 9 vs. 8).

### Example 5

### 5′-ppp-SEQ ID NO: 1 Activates Innate Immunity and Protects Mice from Lethal Influenza Infection

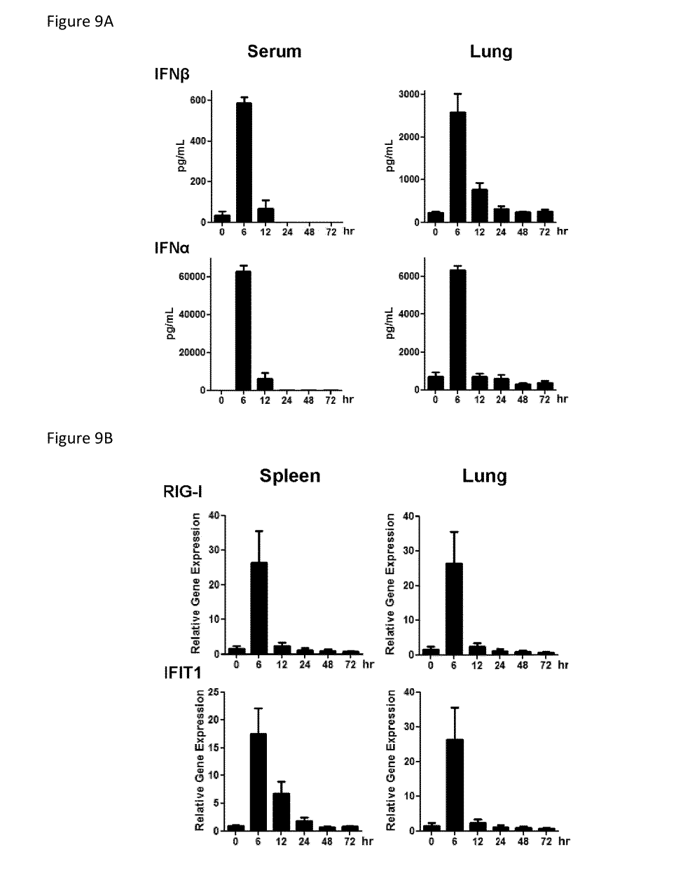

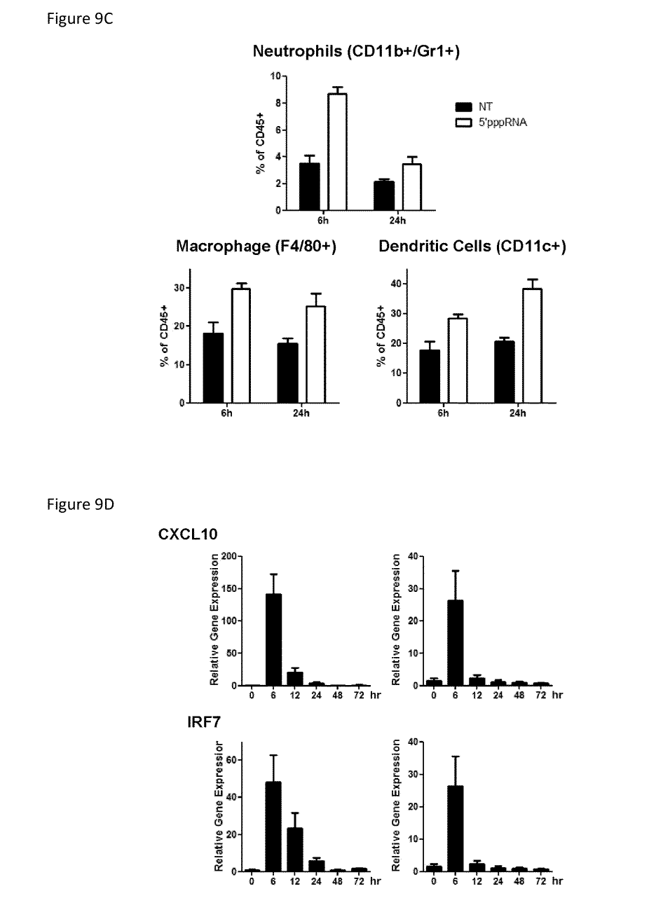

C57BI/6 mice were inoculated intravenously with 5′ppp-SEQ ID NO: 1 in complex with in vivo-jetPEI™ transfection reagent. 5′ppp-SEQ ID NO: 1 stimulated a potent immune response in vivo characterized by IFNα and IFNβ secretion in the serum and lungs (FIG. 9A) as well as antiviral gene up-regulation (FIG. 9B). Following intravenous injection, serum IFNβ levels were increased ^{˜}20-fold compared to basal levels, as early as 6 hours post administration (FIG. 9A top left panel). The immune activation observed in vivo correlated with an early and transient recruitment of neutrophils to the lungs along with a more sustained increase in macrophages and dendritic cells (FIG. 9C).

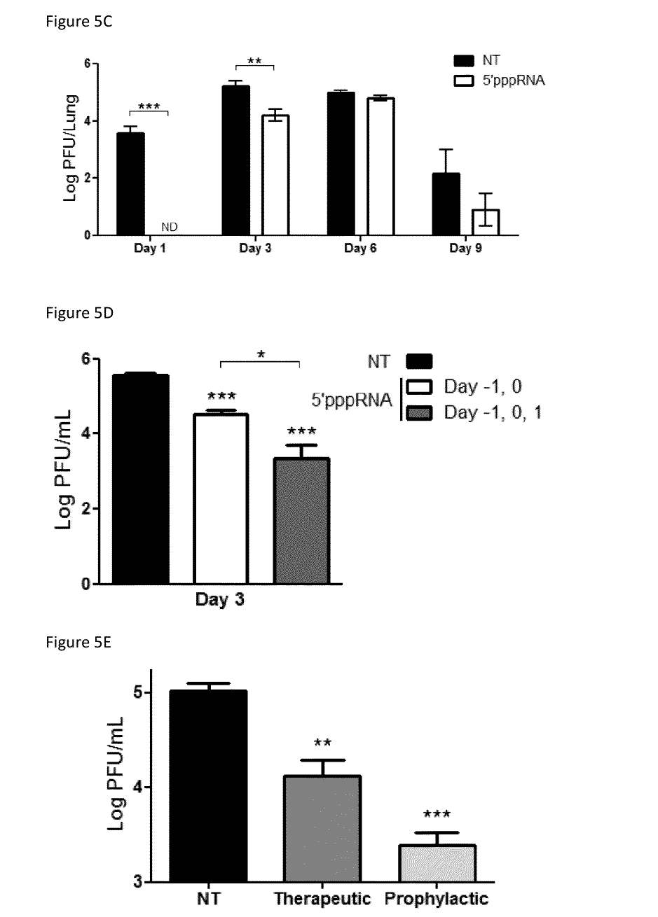

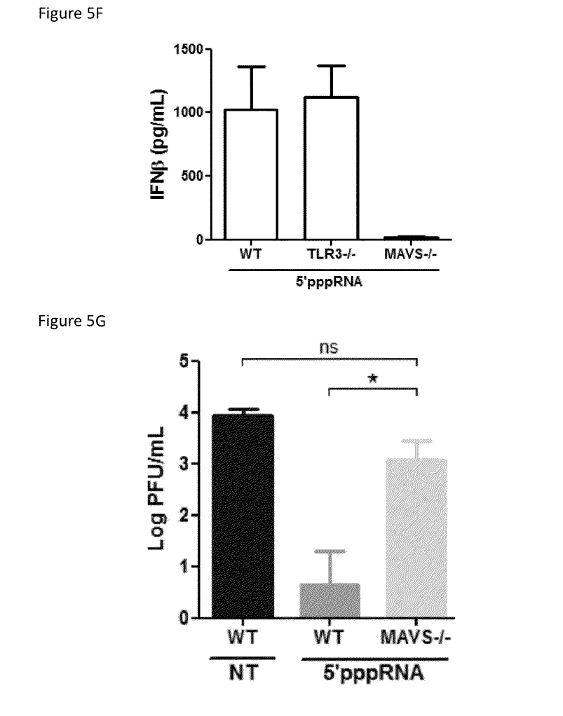

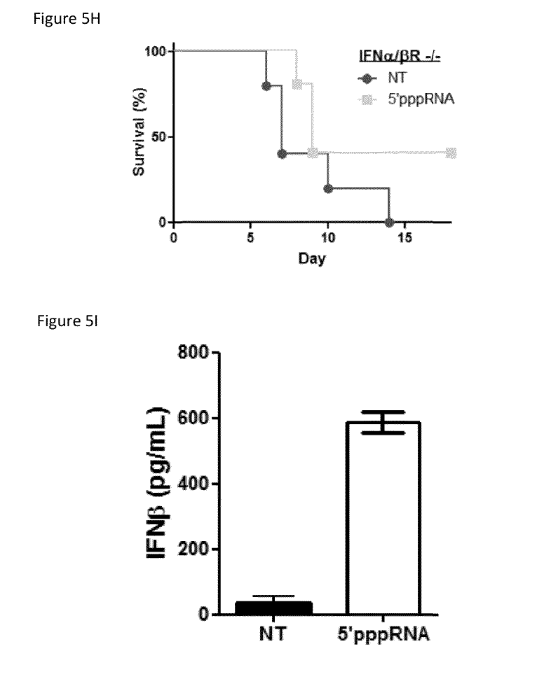

In another experiment, mice were treated with 25 μg of 5′ppp-SEQ ID NO: 1 as described above 24 hours before (day −1), and on the day of infection (day 0) with a lethal inoculum of H1N1 A/PR/8/34 Influenza. All untreated, infected mice succumbed to infection by day 11, but all 5′ppp-SEQ ID NO: 1-treated mice fully recovered (FIG. 5A). Overall weight loss was similar between the two groups (FIG. 5B), although a delay of 2-3 days of the onset of weight-loss was observed in 5′ppp-SEQ ID NO: 1-treated animals. Treated mice fully recovered within 12-14 days (FIG. 5B). Influenza replication in the lungs was monitored by a plaque assay performed throughout the course of infection. Virus titers in the lungs of untreated mice peaked at day 3 post-infection (FIG. 5C) with a decrease in virus titer observed at day 9 post-infection. In the 5′-ppp-SEQ ID NO: 1 treated animals, influenza virus replication in the lungs was inhibited within the first 24-48 hours (FIG. 5C; Day 1). By day 3, virus titers in the lung had increased, although influenza titers were still ^{˜}10-fold lower compared to titers in untreated mice (FIG. 5C; Day 3). By day 9, the 5′ppp-SEQ ID NO: 1 had a sufficiently low viral titer to indicate that they controlled the infection. Continuous administration of 5′ppp-SEQ ID NO: 1 at 24 hour intervals post-infection had an additive therapeutic effect that further delayed viral replication (FIG. 5D; 3 versus 2 doses of 5′ppp-SEQ ID NO: 1). Administration of 5′ppp-SEQ ID NO: 1 therapeutically also controlled influenza viral replication. Administration of 5′ppp-SEQ ID NO: 1 at day 1 and day 2 following infection reduced viral lung titers by ^{˜}10-fold (FIG. 5E).

IFNβ release did not occur in MAVS^{-/-} mice treated with 5′ppp-SEQ ID NO: 1 but did occur in TLR3^{-/-} mice treated with 5′ppp-SEQ ID NO: 1 indicating that IFNβ release by 5′ppp-SEQ ID NO: 1 is dependent on an intact RIG-I pathway (FIG. 5F). MAVS^{-/-} mice treated with 5′ppp-SEQ ID NO: 1 did not control influenza lung titers (5-fold increase vs. wt mice) and the titer was comparable to untreated wt mice (FIG. 5G).

In another experiment, IFNα/βR^{-/-} mice were treated with 5′ppp-SEQ ID NO: 1 and infected with influenza H1N1 virus and compared to untreated infected IFNα/βR^{-/-}. While untreated IFNα/βR^{-/-} animals succumbed to infection, 40% of the animals that received 5′ppp-SEQ ID NO: 1 treatment survived, suggesting that an IFN-independent effect of 5′ppp-SEQ ID NO: 1 provided some protection.

### Example 8

### 5′Ppp-SEQ ID NO: 1 Treatment Limits Influenza-Mediated Pneumonia

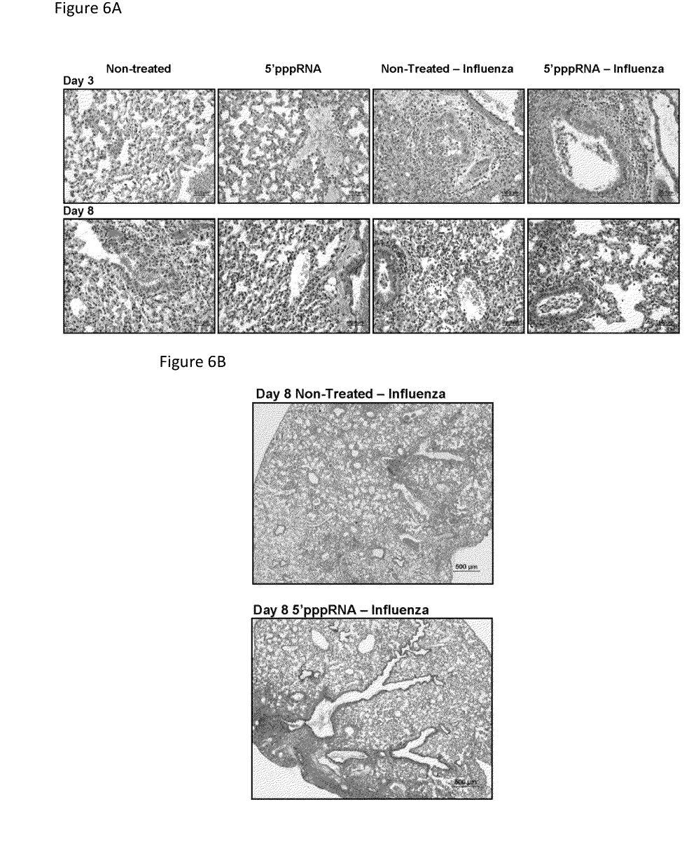

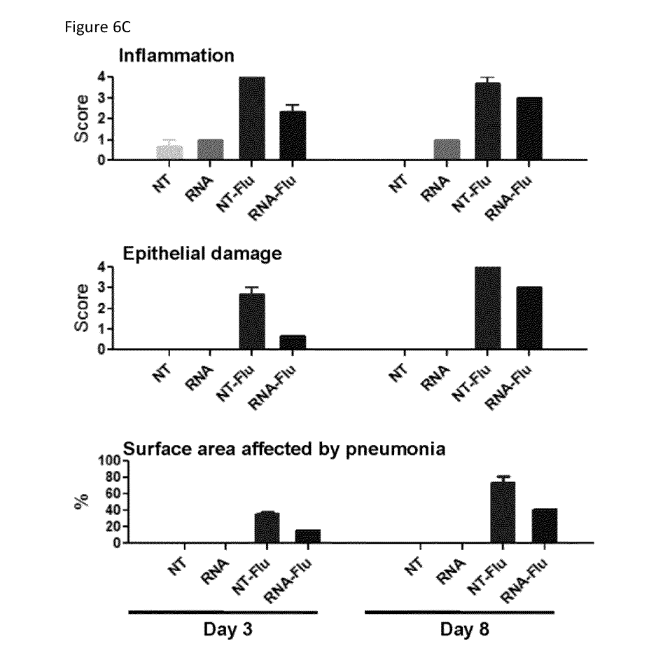

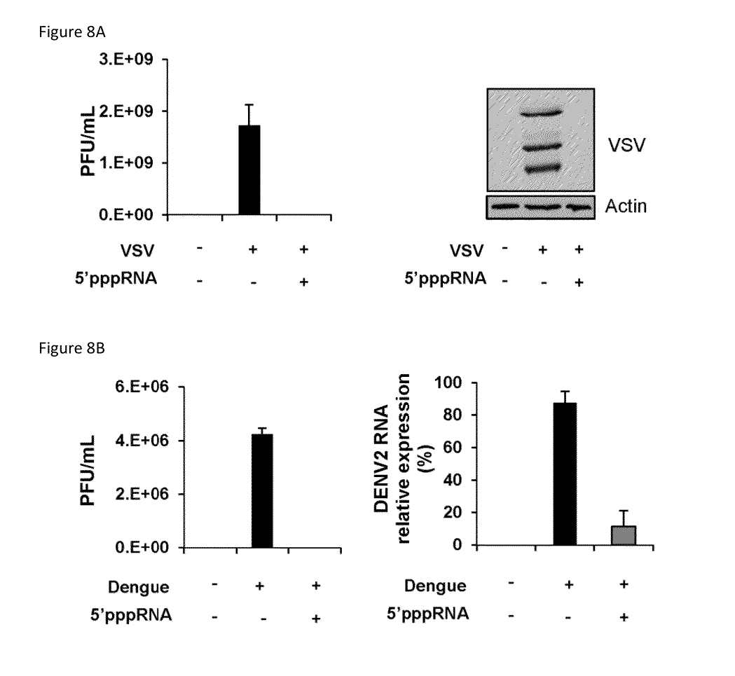

To further evaluate the effect of 5′ppp-SEQ ID NO: 1 administration on influenza-mediated pathology, histological sections of lungs from mice treated with 5′ppp-SEQ ID NO: 1 were compared to untreated mice. 5′ppp-SEQ ID NO: 1 treatment alone (no infection) was characterized by a modest and rare leukocyte-to-endothelium attachment. Mixed leukocyte populations (mononuclear/polymorphonuclear) infiltrated the perivascular space at 24 h after injection but the infiltration resolved and was limited to endothelial cell attachment at 3 and 8 days after intravenous administration (FIG. 6A). Influenza virus infection without treatment with 5′ppp-SEQ ID NO: 1 induced severe and extensive inflammation and oedema in the perivascular space and the bronchial lumen at day 3 post-infection.

In animals infected with Influenza virus and treated with 5′ppp-SEQ ID NO: 1, influenza infection triggered a mild and infrequent inflammation that did not extend to the bronchial lumen at day 3 post-infection. Epithelial degeneration and loss of tissue integrity were more severe in the lungs of untreated, infected animals and correlated with epithelial hyperplasia observed at later times, when compared to the lungs of animals treated with 5′ppp-SEQ ID NO: 1. Inflammation and epithelial damage progressed in untreated mice by day 8 (FIG. 6B), and correlated with the increased viral titer in the lungs described above. Inflammation and epithelial damage was consistently less apparent in influenza infected mice treated with 5′ppp-SEQ ID NO: 1. The surface area of the lungs affected by pneumonia was significantly reduced in 5′ppp-SEQ ID NO: 1-treated mice compared to infected, but untreated mice. On day 3, 16% of the surface area of infected 5′ppp-SEQ ID NO: 1 treated mice was affected by pneumonia while 35% of the surface area of infected untreated mice. By day 8, 41% of the surface area of 5′ppp-SEQ ID NO: 1 treated mice was affected by pneumonia vs 73% of the surface area of infected untreated mice (FIG. 6C; bottom panel). Overall, influenza-mediated pneumonia was less severe in animals administered 5′ppp-SEQ ID NO: 1 before infection with influenza.

### Example 9

### Materials and Methods

In vitro synthesis of 5′ppp-SEQ ID NO: 1:

In vitro transcribed RNA was prepared using the Ambion MEGAscript® T7 High Yield Transcription Kit according to the manufacturer's instruction. The template included two complementary viral sequences operably linked to a T7 promoter that were annealed at 95° C. for 5 minutes and cooled down gradually over night. The in vitro transcription reactions proceeded for 16 hours. 5′ppp-SEQ ID NO: 1 was purified and isolated using the Qiagen miRNA Mini® Kit. An oligoribonucleotide equivalent to SEQ ID NO: 1 lacking a 5′ ppp moiety was purchased from Integrated DNA Technologies, Inc. A secondary structure of 5′ppp-SEQ ID NO: 1 was predicted using the RNAfold WebServer (University of Vienna, Vienna, Austria).

Cell Culture, Transfections, and Luciferase Assays:

A549 cells were grown in F12K media supplemented with 10% FBS and antibiotics. To generate a stable MAVS-negative cell line, a MAVS specific shRNA was used (Xu L G et al, 2005 supra). Plasmids pSuper VISA® RNAi and pSuper® control shRNA were transfected in A549 cells using Lipofectamine 2000® according to the manufacturer's instructions. MAVS-negative cells were selected beginning at 48 hours for approximately 2 weeks in F12K containing 10% FBS, antibiotics, and 2 μg/m; puromycin. Mouse endothelial fibroblasts (MEF's) were grown in DMEM supplemented with 10% FBS, non-essential amino acids, and L-Glutamine. RIG-I^{-/-} MEFS are described in Kato H et al, Immunity 23, 19-28 (2005); (incorporated by reference herein). MDA5^{-/-}, TLR3^{-/-}, and TLR7^{-/-} MEFS are described in Gitlin L et al, Proc Natl Acad Sci USA 103, 8459-3464 (2006) and McCartney S et al, J Exp Med 206, 2967-2976 (2009), both of which are incorporated by reference herein.

Lipofectamine RNAiMax® was used for transfections in A549 according to manufacturer's instructions. For luciferase assays, transfections were performed in wt and RIG-I^{-/-}; wild type, MDA5^{-/-}, TLR3^{-/-}, and TLR7^{-/-} MEFs using Lipofectamine 2000® and jetPRIME®. Plasmids encoding GFP-RIG-I, IRF-7, pRLTK, IFNα4/pGL3 and IFNβ/pGL3 were previously described in Zhao T et al, Nat Immunol 8, 592-600 (2007). The IFNλ1-luciferase reporter is described in Osterlund P I et al, J Immunol 179, 3434-3442 (2007) which is incorporated by reference herein.

MEFs were co-transfected with 200 ng pRLTK reporter (Renilla luciferase for internal control), 200 ng of reporter gene constructs: IFNα4, IFNβ, and IFNλ1, together with 5′ppp-SEQ ID NO: 1 (500 ng/ml) or 100 ng of a plasmid encoding a constitutively active form of RIG-I (ΔRIG-I) (Yoneama M et al Nat Immunol 5, 730-737 (2004), incorporated by reference herein.) IRF7 plasmid (100 ng) was added for transactivation of the IFNα4 promoter. At 24 h after transfection, reporter gene activity was measured by a Promega Dual-Luciferase Reporter Assay according to manufacturer's instructions. Relative luciferase activity was measured as fold induction relative to the basal level of the reporter gene.

Immunoblot Analyses:

Whole cell extracts (40 μg) were separated in 8% acrylamide gel by SDS-PAGE and were transferred to a nitrocellulose membrane at 4° C. for 1 hour at 100 volts in a buffer containing 30 mM Tris, 200 mM glycine and 20% methanol. Membranes were blocked for 1 h at room temperature in 5% dried milk (wt/vol) in PBS and 0.1% Tween-20 (vol/vol) and probed with primary antibodies to IRF3 phosphorylated at Ser396, IRF3, RIG-I, ISG56, STAT1 phosphorylated at Tyr701, STAT1, NS1, IκBα phosphorylated at Ser32, IκBα, NOXA, cleaved Caspase 3, PARP, and β-actin. Antibody signals were detected by chemiluminescence using secondary antibodies conjugated to horseradish peroxidise and an Amersham Biosciences ECL detection kit.

IRF3 Dimerization:

Whole cell extracts were prepared in NP-40 lysis buffer (50 mM Tris pH 7.4, 150 mM NaCl, 30 mM NaF, 5 mM EDTA, 10% glycerol, 1.0 mM Na₃VO₄, 40 mM β-glycerophosphate, 0.1 mM phenylmethylsulfonyl fluoride, 5 μg/ml of each leupeptin, pepstatin, and aproptinin, and 1% Nonidet P-40). Whole cell extracts were then electrophoresed on 7.5% native acrylamide gel, which was pre-run for 30 min at 4° C. The upper chamber buffer was 25 mM Tris at pH 8.4, 192 mM glycine, and 1% sodium deoxycholate and the lower chamber buffer (25 mM Tris at pH 8.4 and 192 mM glycine). Gels were soaked in SDS running buffer (25 mM Tris, at pH 8.4, 192 mM glycine, and 0.1% SDS) for 30 min at 25° C. and were then transferred to nitrocellulose membrane. Membranes were blocked in PBS containing 5% milk (wt/vol) and 0.05% Tween®-20 (vol/vol) for 1 hour at 25° C. and blotted with an antibody against IRF3. Antibody signals were detected by chemiluminescence using secondary antibodies conjugated to horseradish peroxidise and an Amersham Biosciences ECL detection kit.

ELISA:

The release of human IFNα (multiple subunits) and IFNβ in culture supernatants of A549, and murine IFNβ in mouse serum were measured using the appropriate ELISA kits from PBL Biomedical Laboratories according to manufacturer's instructions.

Primary Cell Isolation:

PBMCs were isolated from freshly collected human blood using a Cellgro® Lymphocyte Separation Medium according to manufacturer's instructions. After isolation, total PBMCs were frozen in heat-inactivated FBS with 10% DMSO. On experimental days, PBMCs were thawed, washed and placed at 37° C. for 1 hour in RPMI with 10% FBS supplemented with Benzonaze® nuclease to prevent cell clumping.

Virus Production and Infection

VSV-GFP, which harbors the methionine 51 deletion in the matrix protein-coding sequence (Stojdl D et al, Cancer Cell 4, 263-275 (2003) was grown in Vero cells, concentrated from cell-free supernatants by centrifugation, and titrated by a standard plaque assay as described previously in Tumilasci V F et al, J Virol 82, 8487-8499 (2008), incorporated by reference herein. The recombinant vaccinia-GFP virus VVE3L-REV), a revertant strain of the E3L deletion mutant is described in Myskiw C et al, J Virol 85, 12280-12291 (2011) and Arseniob J et al, Virology 377, 124-132 (2008).

Dengue virus serotype 2 (DENV-2) strain New Guinea C was grown in C6/36 insect cells for 7 days. Cells were infected at a MOI of 0.5, and 7 days after infection, cell supernatants were collected, clarified and stored at −80° C. Titers of DENV stocks were determined by serial dilution on Vero cells and intracellular immunofluorescent staining of DENV E protein at 24 hours post-infection. Titer is given as infectious units per ml. In infection experiments, both PBMCs and A549 cells were infected in a culture media without FBS for 1 hour at 37° C. and then incubated with complete medium for 24 hours prior to analysis.

HIV-GFP virus is an NL4-3 based virus designed to co-express Nef and eGFP from a single bicistronic RNA. HIV-GFP particles were produced by transient transfection of pBR43IeG-nef+ plasmid into 293T cells as described in Schindler M et al, J Virol 79, 5489-5498 (2005) and Schindler M et al, J Virol 77, 10548-10556 (2003), both of which are incorporated by reference herein. 293T cells were transfected with 22.5 μg of pBR43IeG-nef+ plasmid by polyethylenimine precipitation. Media was replaced 14 to 16 hours post-transfection, viral supernatants were harvested 48 hours later, cleared by low-speed centrifugation and filtered through a 0.45 μm low binding protein filter. High-titer viral stocks were prepared by concentrating viral supernatants 100-fold through filtration columns. These were then stored at −80° C. Viral titers were determined by p24 level (ELISA) and TCID50. A set of 10-fold serial dilutions of concentrated viral supernatants were used to infect PBMCs pre-activated for 3 days with 10 μg/ml of PHA. Four days after infection half the media was replaced. Seven days after infection, supernatants were harvested and titered by ELISA. TCID50T was calculated by the Reed-Muench method.

CD14⁺ monocytes were negatively selected using the EasySep® Human Monocytes Enrichment Kit as per manufacturer's instructions. Isolated cells were transfected with 5′ppp-SEQ ID NO: 1 (100 ng/ml) using Lyovec (Invitrogen) according to the manufacturer's protocol. Supernatants were harvested 24 hours after stimulation and briefly centrifuged to remove cell debris. CD4⁺ T cells were isolated using EasySep® Human CD4⁺ T cells Enrichment Kit according to the manufacturer's instructions. Purified CD14⁺ monocytes and CD4⁺ T cells were allowed to recover for 1 hour in RPMI containing 10% FBS at 37° C. with 5% CO₂ before experiments. For HIV infection, anti-CD3 antibodies at 0.5 μg/ml were immobilized for 2 hours in a 24-well plate. CD4⁺ T cells were then added along with an anti-CD28 antibody (1 μg/ml) to allow activation of T cells for 2 days. After activation, cells were incubated for 4 hours with supernatant of monocytes stimulated with 5′ppp-SEQ ID NO: 1 and infected with HIV-GFP at an MOI of 0.1. Supernatant from the monocytes was left for another 4 h before adding complete medium.

HCV RNA was synthesized using the Ambion MEGAscript® T7 High Yield Transcription Kit using linearized pJFH1 DNA as a template. Huh7 cells were electroporated with 10 mg of HCV RNA. At 5 days post-transfection, supernatants containing HCV (HCVcc) were collected, filtered (0.45 μm) and stored at −80° C. Huh7 or Huh7.5 cells were pre-treated with 5′-ppp-SEQ ID NO: 1 (10 ng/ml) for 24 h. Cell culture supernatants containing soluble factors induced following 5′-ppp-SEQ ID NO: 1 treatment were removed and kept aside during infection. Cells were washed once with PBS and infected with 0.5 ml of undiluted HCVcc for 4 hours at 37° C. After infection, supernatant from 5′ppp-SEQ ID NO: 1 treated cells was added back. At 48 hours post infection, whole cell extracts were prepared and the expression of HCV NS3 protein was detected by Western blot.

Influenza H1N1 strain A/Puerto Rico/8/34 was amplified in Madin-Darby canine kidney (MDCK) cells and virus titer determined by standard plaque assay (Szretter K J et al, Curr Protoc Microbiol Chapter 15.1 (2006), incorporated by reference herein.) Cells were infected in 1 ml medium without FBS for 1 hour at 37° C. Inoculum was aspirated and cells were incubated with complete medium for 24 hours, unless otherwise indicated, prior to analysis. For viral infections, supernatants containing soluble factors induced by treatment with 5′ppp-SEQ ID NO: 1 were removed and kept aside during infection. Cells were washed once with PBS and infected in a small volume of medium without FBS for 1 h at 37° C.; then supernatant was then added back for the indicated period of time.

Flow Cytometry:

The percentage of cells infected with VSV, Vaccinia and HIV was determined based on GFP expression. The percentage of cells infected with Dengue was determined by standard intracellular staining. Cells were stained with a mouse IgG2a monoclonal antibody specific for DENV-E-protein (clone 4G2) followed by staining with a secondary anti-mouse antibody coupled to PE. PBMCs infected with DENV2 were first stained with anti-human CD14 AlexaFluor® 700 Ab. Cells were analyzed on a LSRII® flow cytometer. Compensation calculations and cell population analysis were done using FACS® Diva.

In Vivo Administration of 5′Ppp-SEQ ID NO: 1 and Influenza Infection Model:

C57BI/6 mice (8 weeks) were obtained from Charles River Laboratories. MAVS^{-/-} mice on a mixed 129/SvEv-C57BI/6 background were obtained from Z. Chen (The Howard Hughes Medical Institute, US). TLR3^{-/-} mice were obtained from Taconic. For intra-cellular delivery, 25 ug of 5′ppp-SEQ ID NO: 1 was complexed with In vivo-JetPEI® at an N/P ratio of 8 as per manufacturer's instructions and administered intravenously via tail vein injection. Unless otherwise indicated, 5′ppp-SEQ ID NO: 1 was administered on the day prior to infection (Day −1) and also on the day of infection (Day 0). Mice infected intra-nasally with 500 pfu of Influenza A/PR/8/34 under 4% isoflurane anesthesia. For viral titers, lungs were homogenized in DMEM (20% wt/vol) and titers were determined by standard plaque assay as previously described in Szretter K J et al, 2006 supra. Confluent Madin-Darby Canine Kidney Cells (MDCK) were incubated with 250 μL of serial 10-fold dilutions of homogenized lung sample for 30 minutes. The sample was aspirated, and cells overlaid with 3 ml of 1.6% agarose in DMEM. Plaques were fixed and counted 48 hours later.

Histology and Pathology:

All five lobes of the lungs were collected and fixed in neutral-buffered formalin for 24 hours. The tissues were paraffin-embedded and 4 μm sections were prepared using a microtome. Hematoxylin and eosin staining (H&E) were performed using standard protocols and analyzed by an independent veterinary pathologist.

Microarray Analysis:

A549 cells were stimulated with either 5′ppp-SEQ ID NO: 1 (10 ng/ml) or IFNα-2b (100 IU/ml or 1000 IU/ml) for designated times. Cells were collected and lysed for RNA extraction. Reverse transcription reactions were performed to obtain cDNAs which were hybridized to the Illumina Human HT-12 version 4 Expression BeadChip® according to the manufacturer's instructions, and quantified using an Illumina iScan® System. The data were collected with Illumina GenomeStudio® software.

Arrays displaying unusually low median intensity, low variability, or low correlation relative to the bulk of the arrays were not analyzed. Quantile normalization was applied, followed by a log₂ transformation using the Bioconductor® package LIMMA. Batch effect subtraction was done using the ComBat procedure (http://dx.doi.org/10.1093/biostatistics/kxj037). Missing values were imputed with R package impute (http://cran.r-project.org/web/packages/impute/index.html). The LIMMA package (Smyth G K et al, in Bioinformatics and Computational Biology Solutions using R and Bioconductor, 397-420, NY, Springer (2005), incorporated by reference herein.) was used to fit a linear model to each probe and to perform a moderated Student's t test on differentially expressed genes.

Genes with significant differential expression levels were identified using Bioconductor LIMMA package with ≧2.5 fold change (up or down) for the kinetic assay and ≧2.0 fold change; raw (nominal) p-value ≦0.05 for the comparison to IFNα-2b, the false discovery rate (FDR) adjusted P value <0.05 or FDR level set at 5%. Gene expression within each heatmap is represented as gene-wise standardized expression (Z-score), with |FC|>2.5 or 2.0 for the kinetic assay and p-value <0.05 and FDR <5% chosen as the significant levels. The expected proportions of false positives (FDR) were estimated from the unadjusted p-value using the Benjamini and Hochberg method (Benjamini Y A, H Yosef, J R Stat Soc Series B Stat Methodol 57, 289-300 (1995), incorporated by reference herein.

All network analysis was done with Ingenuity Pathway Analysis. The input data includes genes whose expression levels meet the following criteria: ≧2.5 fold change (up or down) for the kinetic assay and ≧2.0 fold change; raw (nominal) p-value ≦0.05 for the comparison to IFNα-2b. The genes in the data were mapped to the Ingenuity Pathway knowledge base with different colors (red: up-regulated; green: down-regulated) based on Entrez Gene IDs. The significance of the association between the dataset and the canonical pathway was measured in two ways: (1) A ratio of the number of genes from the dataset that map to the pathway divided by the total number of genes that map to the canonical pathway was displayed; (2) overrepresentation Fisher's exact test was used to calculate a p-value determining the probability that the association between the genes in the dataset and the canonical pathway is explained by chance alone. The pathways were ranked with −log p values.

Quantitative real-time PCR: Total RNA was isolated from cells using a Qiagen RNeasy® Kit. 1 μg of RNA was reverse transcribed using a High-Capacity cDNA Reverse Transcription Kit from Applied Biosystems according to manufacturer's instructions. Parallel reactions without reverse transcriptase were included as negative controls. The relative amount of an intracellular RNA of interest was quantified by real-time PCR on a real-time PCR system and expressed as a fold change using SYBR Green according to the manufacture's protocol. All data presented are relative quantification with efficiency correction based on the relative expression of target genes versus GAPDH as a housekeeping gene.

### Example 8

### 5′ppp-SEQ ID NO: 1 Inhibits DENV Infection

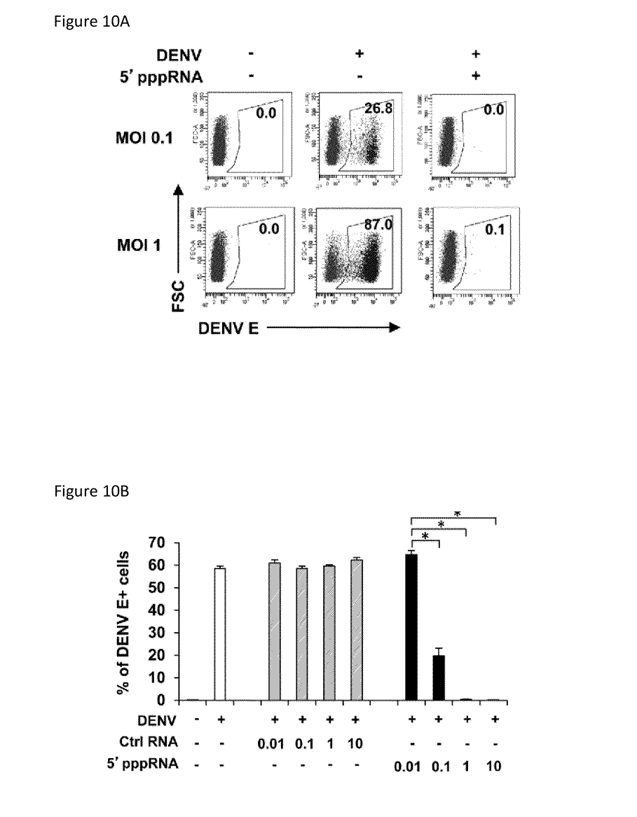

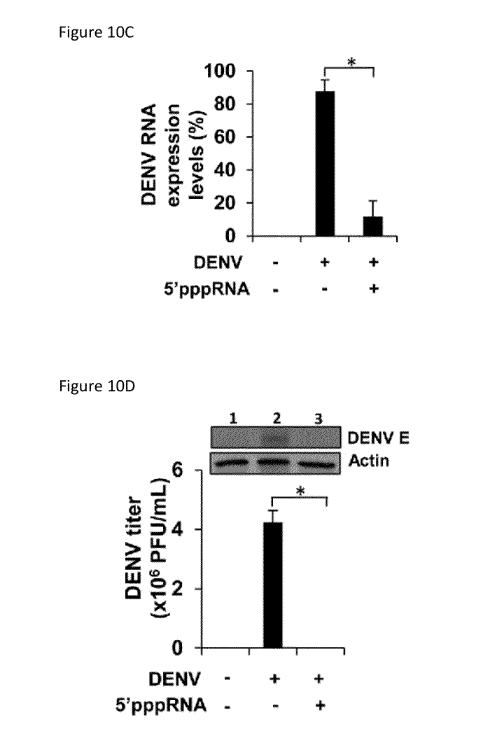

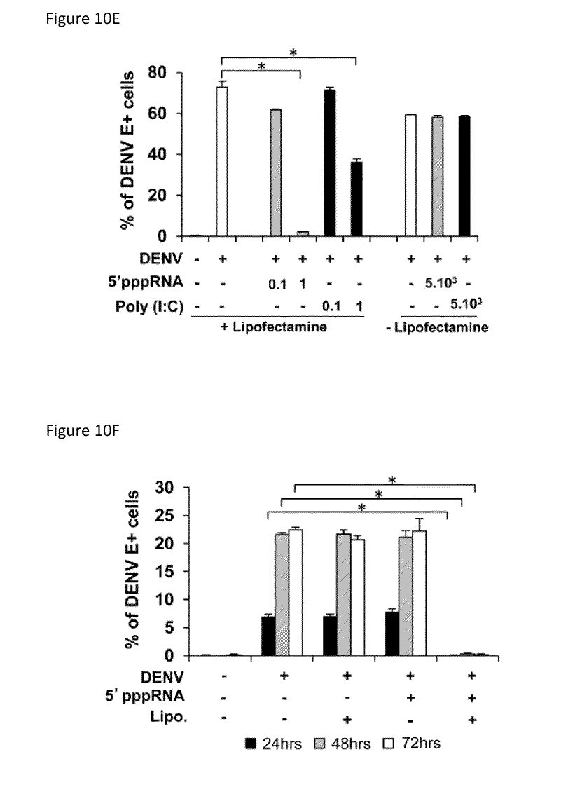

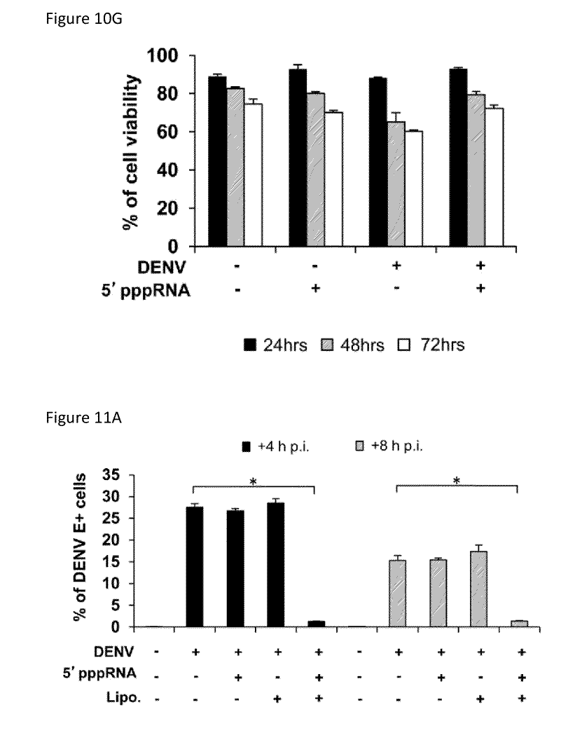

5′ppp-SEQ ID NO:1 inhibits DENV infection. To determine the capacity of the 5′ppp-SEQ ID NO:1 RIG-I agonist to induce a protective antiviral response to DENV infection, A549 cells were challenged with DENV at different multiplicities of infection (MOI); infection. Replication was monitored by flow cytometry, RT-qPCR, plaque assay, and immunoblotting (FIG. 10A to 10F). DENV established infection in A549 cells. The infection was completely abrogated in cells pretreated with 1 ng/ml of 5′ppp-SEQ ID NO: 1 (FIG. 10A). A similar antiviral effect was observed at higher concentrations of 5′ppp-SEQ ID NO: 1 (10 ng/ml). The antiviral effect was dependent on the 5′ppp-moiety because transfection of cells with the identical RNA sequence lacking the 5′ ppp did not prevent DENV infection (FIG. 10B). Pretreatment of cells with 5′ppp-SEQ ID NO: 1 also led to an 8.5-fold decrease in DENV RNA synthesis (FIG. 10C). Release of infectious DENV was completely suppressed by 5′ppp-SEQ ID NO: 1 treatment (4.3×10⁶ PFU/ml in untreated cells versus undetectable in 5′ppp-SEQ ID NO: 1 treated cells) (FIG. 10D). This led to a complete inhibition of DENV E protein expression (FIG. 10D, lane 3). To compare the effect of 5′ppp-SEQ ID NO: 1 to that of the dsRNA ligand poly(I:C), A549 cells were pretreated with 5′ppp-SEQ ID NO: 1 or poly(I:C) (0.1 to 1 ng/ml) and subsequently challenged with DENV (FIG. 10E). Treatment with 1 ng/ml of 5′ppp-SEQ ID NO: 1 almost completely suppressed DENV infection. At the same concentration, only a 1.8-fold decrease of the number of DENV-infected cells was observed with poly(I:C) treatment (FIG. 10E). Cytosolic delivery of dsRNA by transfection was required in A549 cells, as demonstrated by the absence of a protective antiviral effect in cells in medium to which 5 μg/ml of 5′ppp-SEQ ID NO: 1 or poly(I:C) had just been added (FIG. 10E).

To determine whether pretreatment with 5′ppp-SEQ ID NO: 1 maintained a protective effect, A549 cells were transfected with 5′ppp-SEQ ID NO: 1 prior to DENV challenge and the virus was allowed to replicate up to 72 h post infection (FIG. 10F). The combination treatment completely inhibited DENV infection at all time points for up to 72 h post infection (FIG. 10F). The viability of uninfected cells and cells protected from infection by 5′ppp-SEQ ID NO: 1 was indistinguishable (FIG. 10G). Altogether, these results demonstrate the antiviral potential of 5′ppp-SEQ ID NO: 1 against DENV infection in nonimmune cells.

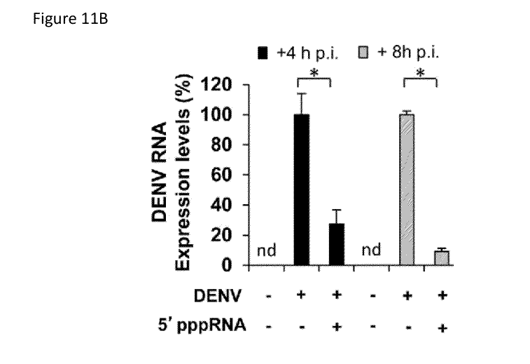

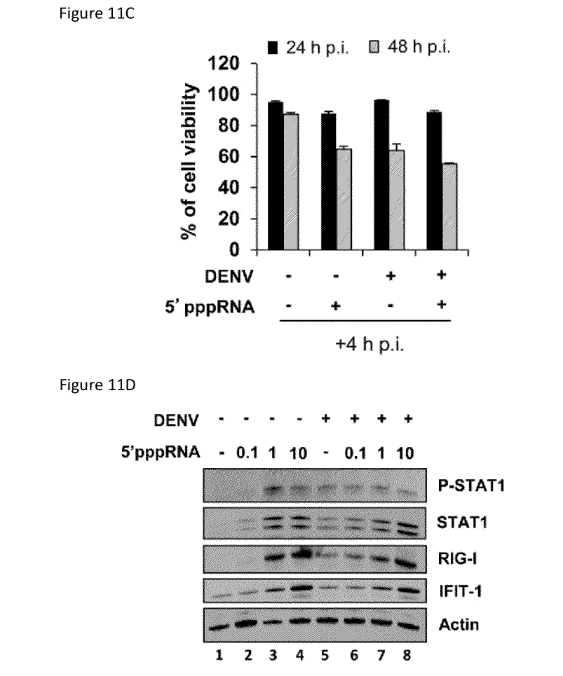

To assess the potential of 5′ppp-SEQ ID NO: 1 as a postinfection treatment, A549 cells were first infected with DENV, subsequently treated with 5′ppp-SEQ ID NO: 1 at 4 h and 8 h after infection, and analyzed 48 h later to detect DENV infection. Infection was almost completely inhibited even when cells were treated at 8 hours post infection, as shown by the 12.4-fold reduction of the number of DENV-infected cells (FIG. 11A). This suggests that as DENV replicates over time 5′ppp-SEQ ID NO: 1 prevents further spread of the virus by protecting uninfected cells and clearing virus from infected cells. The observed effects of 5′ppp-SEQ ID NO: 1 on DENV infection were confirmed by RT-qPCR, yielding a 3.6-fold (+4 hours) and 10.8-fold (+8 hour) decrease in DENV viral RNA levels at 48 h post infection. (FIG. 11B). Cell viability was not significantly affected by a 24-h 5′ppp-SEQ ID NO: 1 treatment and an approximate 20% decrease in viability was observed at 48 h p.i. in cells protected from infection by 5′ppp-SEQ ID NO: 1 (FIG. 11C).

To investigate the antiviral response triggered by 5′ppp-SEQ ID NO: 1, various signaling parameters were monitored by immunoblotting and RT-qPCR in cells treated with increasing doses of 5′ppp-SEQ ID NO: 1 in the presence or absence of DENV infection (FIGS. 11D and 11E). Interferon signaling was detected by immunoblotting in 5′ppp-SEQ ID NO: 1 treated cells, both in the presence or absence of DENV, as demonstrated by increased STAT1 Tyr701 phosphorylation and ISG expression of STAT1, RIG-I, and IFIT1 (FIG. 11D, lanes 2 to 8). Although DENV can evade the host innate response, a significant inhibition of IFN signaling was not observed based on the expression of antiviral markers STAT1, RIG-I, and IFIT1 in infected or uninfected cells (FIG. 11D, lanes 2 to 8).

5′ppp-SEQ ID NO: 1 treatment elicited a strong antiviral response in uninfected and DENV-infected A549 cells (FIG. 11D), and delivery of 5′ppp-SEQ ID NO: 1 at 4 hours post infection potently stimulated type I IFN and inflammatory responses via the upregulation of genes, such as those of IFN-α, IFN-β, IL-6, and IL-1α (FIG. 11E).

### Example 9

### 5′ppp-SEQ ID NO: 1 Restricted DENV Infection Requires an Intact RIG-I Pathway

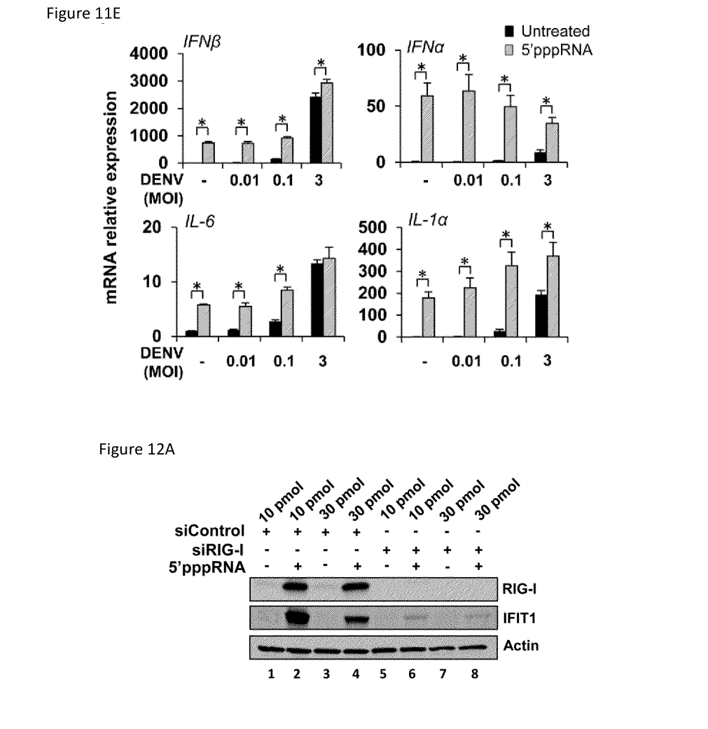

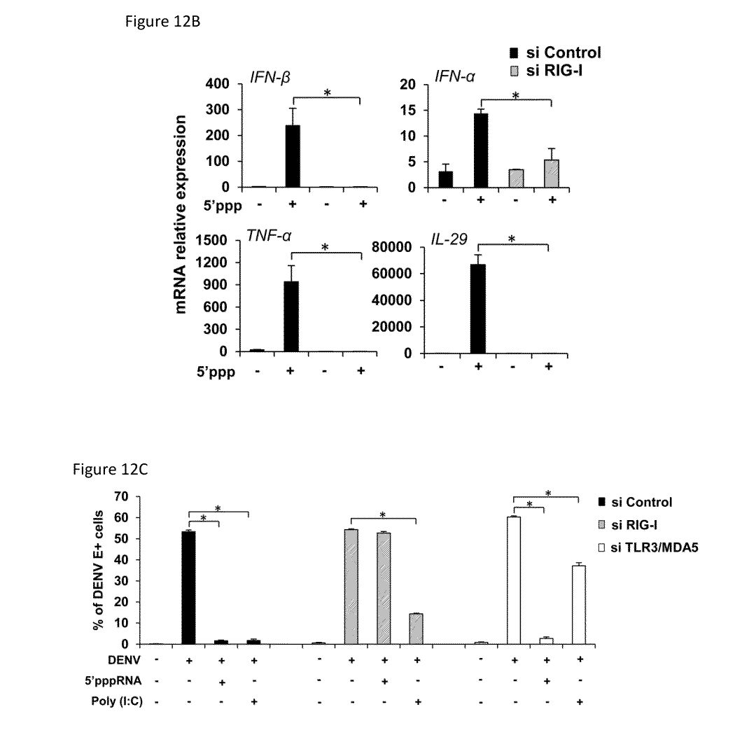

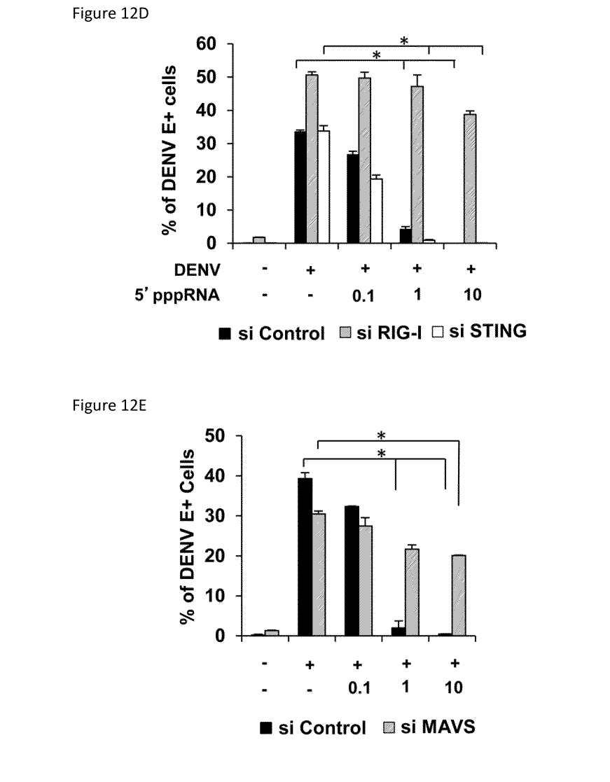

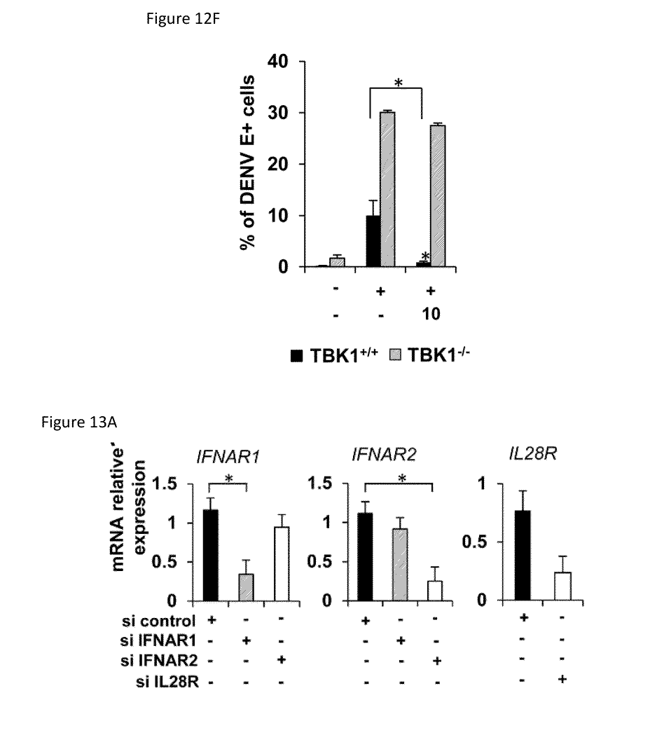

Introduction of RIG-I siRNA (10 and 30 pmol) into A549 cells severely reduced RIG-I as well as IFIT1 induction in response to 5′ppp-SEQ ID NO: 1 treatment (FIG. 12A, lanes 5 to 8). Induction of the type I and type III IFNs, as well as the inflammatory response, were all dependent on intact RIG-I signaling, since the mRNA levels of IFN-α, IFN-β, IL-29, and tumor necrosis factor alpha (TNF-α) were drastically decreased in the absence of RIG-I expression (FIG. 12B). To explore the respective involvement of RIG-I, TLR3, and MDA5 in the 5′ppp-SEQ ID NO: 1 mediated anti-DENV effect, the expression of these immune sensors was knocked down in A549 cells by siRNA (FIG. 12C). While impairing RIG-I expression completely suppressed the 5′ppp-SEQ ID NO: 1-mediated antiviral effect, this was not the case upon knockdown of TLR3/MDA5 (FIG. 12C). The efficacy of poly(I:C) in preventing DENV infection was reduced to a larger extent in the absence of TLR3/MDA5 than in the absence of RIG-I, suggesting a predominant role for TLR3/MDA5 in mediating poly(I:C) antiviral effect in A549 cells (FIG. 12C). To demonstrate that the antiviral activity of 5′ppp-SEQ ID NO: 1 against DENV relies on a functional RIG-I axis, the expression of RIG-I, STING, MAVS, and TBK1 was depleted in A549 cells using specific siRNAs. In addition, suitable knockout MEFs were used (FIG. 12D, 12E, and 12F). Following 5′ppp-SEQ ID NO: 1 treatment, DENV viral replication was assessed by flow cytometry. Whereas about 35% of A549 cells were infected with DENV in the untreated population, the absence of RIG-I led to a 1.5-fold increase in the number of infected cells (FIG. 12D). Transient knockdown of RIG-I resulted in the abrogation of the protective response induced by 5′ppp-SEQ ID NO: 1 in control cells (FIG. 12D), whereas the absence of STING did not affect DENV infection and did not significantly reduce the 5′ppp-SEQ ID NO: 1-induced antiviral response (FIG. 12D). Similar results were observed with A549 cells depleted for the mitochondrial adaptor MAVS. Depletion of MAVS strongly reduced the 5′ppp-SEQ ID NO: 1-mediated protective antiviral response (FIG. 12E). Finally, TBK1-deficient MEFs were more susceptible to DENV infection than wild-type MEFs and were not responsive to 5′ppp-SEQ ID NO: 1 treatment, as demonstrated by the high level of DENV infection (FIG. 12F). In conclusion, 5′ppp-SEQ ID NO: 1 treatment efficiently generates a RIG-I/MAVS/TBK1-dependent antiviral response that limits DENV infection in vitro.

### Example 10

### 5′ppp-SEQ ID NO: 1 Generates an IRF3-Dependent and IFNAR/STAT1-Independent Antiviral Protective Effect

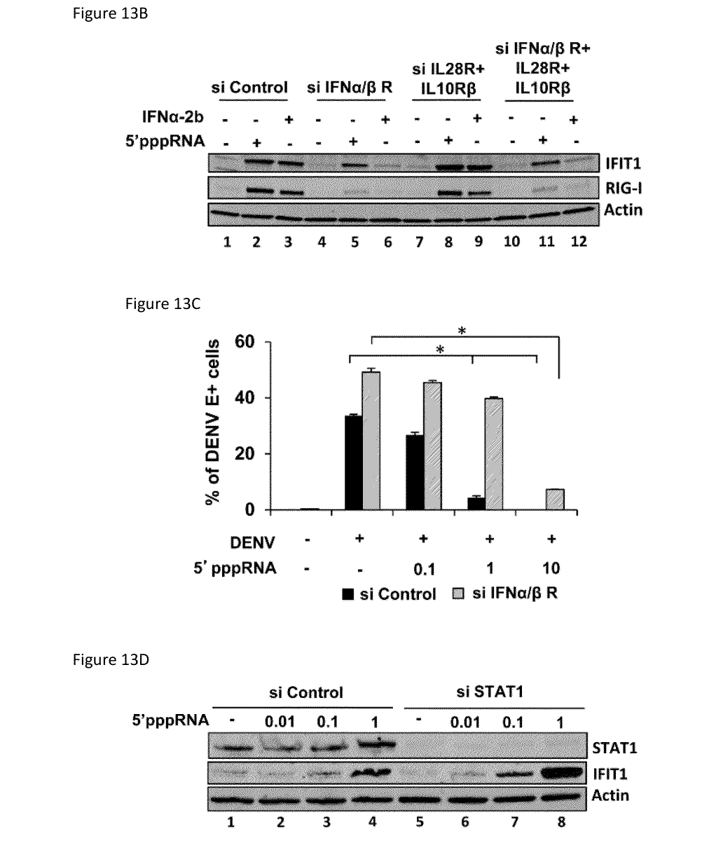

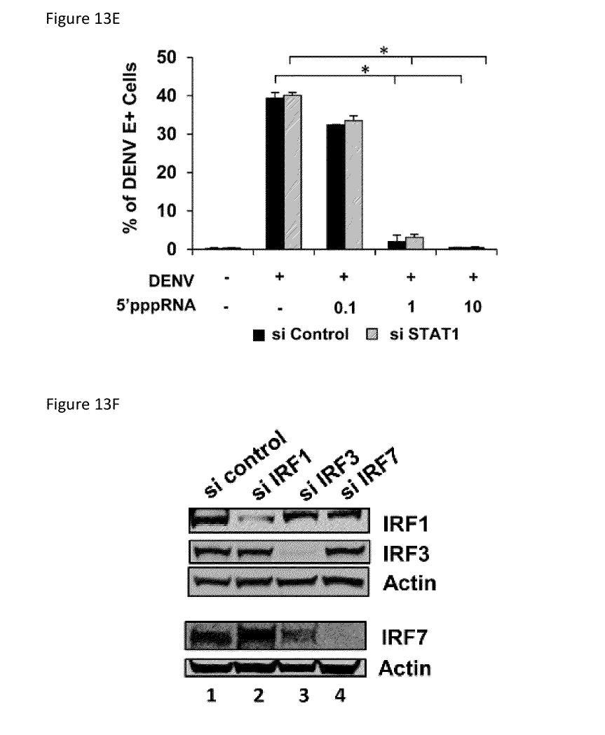

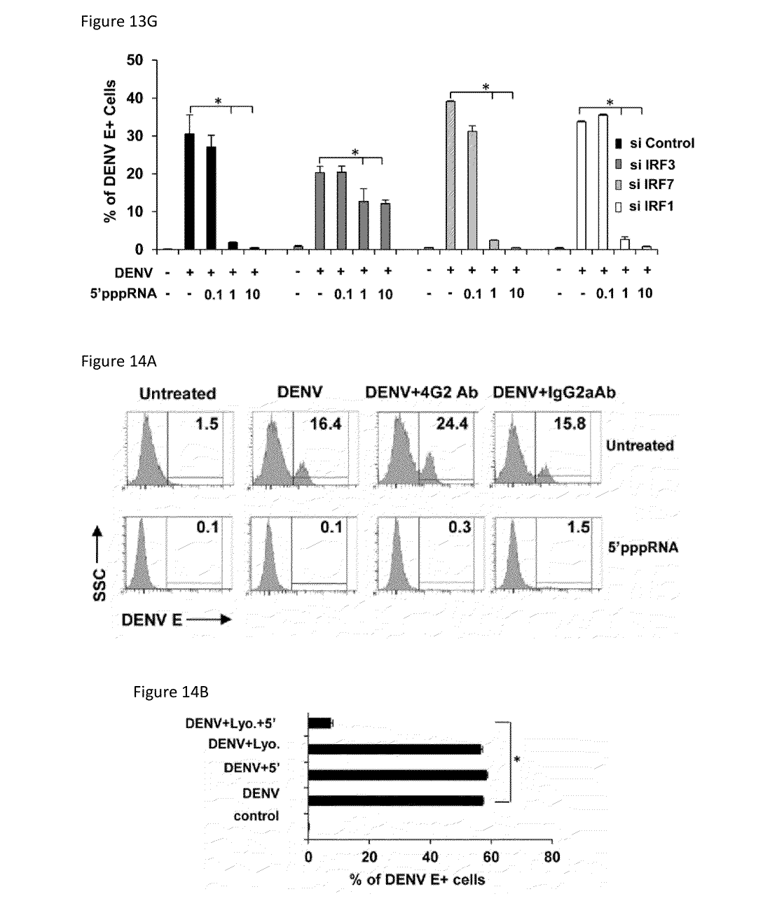

To determine whether the potent RIG-I activation brought about by 5′ppp-SEQ ID NO: 1 could compensate for the type I and type III IFN response, expression of the type I IFN receptor (IFN-α/βR) as well as the type III IFN receptor (IL-28R plus IL-10Rβ) was knocked down using siRNA in A549 cells (FIGS. 13A, 13B and 13C). Expression of both type I and III IFN receptor was efficiently reduced, as shown by the downregulation of IFNAR1 (IFN α/βRα chain), IFNAR2 (IFN-α/βRα chain), and IL-28R mRNA expression levels (FIG. 13A). Furthermore, knockdown of type I IFN signaling was highly efficient, as demonstrated by the reduction of IFIT1 and RIG-I induction following IFN-α2b stimulation (6.2-fold reduction of IFIT1 versus control siRNA [siCTRL]; FIG. 13B, lane 3 versus lane 6). Knocking down the type III IFN receptor did not interfere with the ability of 5′ppp-SEQ ID NO: 1 and IFN-α2b to induce IFIT1 and RIG-I expression (FIG. 13B, lanes 2 and 3 versus lanes 8 and 9).

Induction of IFIT1 but not RIG-I was only partially reduced following 5′ppp-SEQ ID NO: 1 treatment in the absence of type I IFN receptor (1.6-fold reduction of IFIT1 versus siCTRL; FIG. 13B, lane 2 versus lane 5), suggesting that certain ISGs were upregulated by 5′ppp-SEQ ID NO: 1 in an IFN-independent manner. Knocking down expression of both type I and type III IFN receptors did not limit IFIT1 induction by 5′ppp-SEQ ID NO: 1, as the increase of IFIT1 was only reduced 1.9 times compared to the siRNA control (FIG. 13B). This type I and III IFN-independent activation of the innate system was sufficient to suppress DENV infection in A549 cells stimulated with a higher (10 ng/ml) but not a low dose (0.1 to 1 ng/ml) of 5′ppp-SEQ ID NO: 1 (FIG. 13C). To further confirm that type I IFN signaling was not necessarily required to mediate an immune response to 5′ppp-SEQ ID NO: 1, STAT1 was depleted in A549 cells using siRNA (FIG. 13D, lanes 5 to 8). The increased expression of IFIT1 following 5′ppp-SEQ ID NO: 1 treatment was not impacted by the absence of the STAT1 transcription factor (FIG. 13D, lanes 2 to 4 versus lanes 6 to 8). The STAT1-independent induction of the antiviral response was sufficient to block DENV infection in A549 cells stimulated with a high 5′ppp-SEQ ID NO: 1 concentration (FIG. 13E). Finally, to determine which IRF transcription factor downstream of RIG-I was involved in the antiviral protective effect, IRF1, IRF3, and IRF7 expression was knocked down using siRNA (FIG. 13F). Depletion of these different transcription factors was highly efficient, as shown in FIG. 13F. Only IRF3 knockdown resulted in inhibition of the protective antiviral response generated by 5′ppp-SEQ ID NO: 1 treatment. Indeed, the absence of either IRF1 or IRF7 did not impair 5′ppp-SEQ ID NO: 1-mediated antiviral protection (FIG. 13G). Altogether, these data demonstrate that the 5′ppp-SEQ ID NO: 1-mediated anti-DENV effect in vitro is largely independent of the type I or type III IFN responses but requires the activation of a functional RIG-I/IRF3 axis to mediate its protective effect.

### Example 11

### A Protective Antiviral Response Against DENV in Primary Human Myeloid Cells

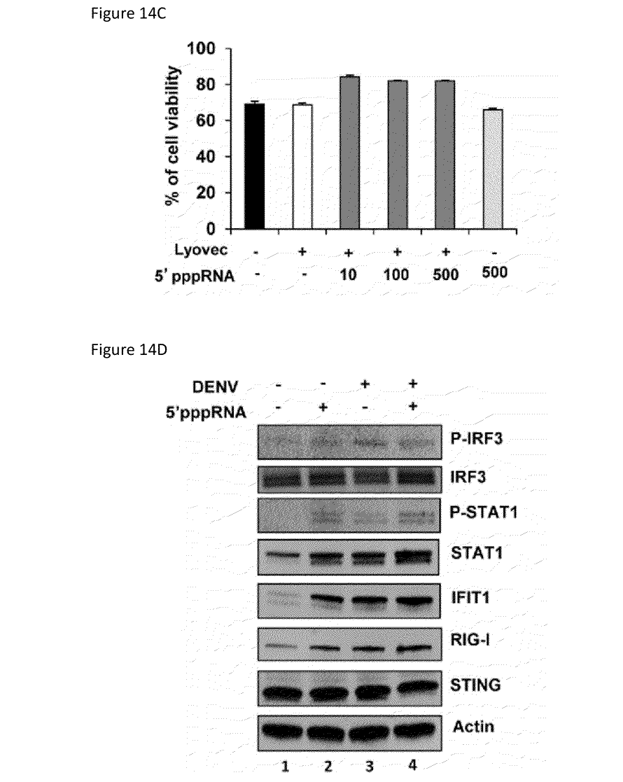

Cells of the myeloid lineage, including monocyte/macrophages and dendritic cells, are the primary target cells for DENV infection among human peripheral blood mononuclear immune cells. Severe and potentially lethal manifestations associated with secondary DENV infection are often related to antibody-dependent enhancement (ADE) of infection. To address the impact of 5′ppp-SEQ ID NO: 1 on ADE-mediated DENV infection, we demonstrated, using isolated human monocytes, that anti-DENV E 4G2 antibody increased DENV infectivity from 16.4% to 24.4% (FIG. 14A), whereas a control isotype IgG2a antibody did not significantly increase viral infectivity (FIG. 14A). Both primary and ADE DENV infections were completely suppressed by 5′ppp-SEQ ID NO: 1 treatment (16.4% and 24.4% in untreated cells versus 0.1% and 0.3% in 5′ppp-SEQ ID NO: 1-treated cells, respectively).

Similarly, in primary human MDDC, which are highly permissive to DENV, infection decreased 8.4-fold in the presence of 5′ppp-SEQ ID NO: 1 in combination with Lyovec (FIG. 14B), and cell viability was not affected by increasing concentrations of 5′ppp-SEQ ID NO: 1 (FIG. 14C). MDDC treated with 5′ppp-SEQ ID NO: 1 at 4 hours post infection. were assessed for markers of activation of the innate immune response (FIG. 14D). Increased levels of phosphorylated IRF3 and STAT1 were observed, and a 2- to 10-fold increase in the expression of ISG RIG-I and IFIT-1 following 5′ppp-SEQ ID NO: 1 treatment were observed (FIG. 14D, lane 2). A similar response was observed with DENV infection alone (FIG. 14D, lane 3). The innate DNA sensor STING was known to be cleaved and inactivated by DENV N52/3 protease. In the experiments disclosed herein, STING expression was not modulated by 5′ppp-SEQ ID NO: 1 or DENV infection alone (FIG. 14D, lane 2 and 3). Also, postinfection treatment with 5′ppp-SEQ ID NO: 1 moderately increased the levels of the following markers of the innate immune response compared to virus alone: phospho-STAT1 (3-fold increase), STAT1 (1.4-fold increase), IFIT1 (1.3-fold increase), and RIG-I (1.3-fold increase) (FIG. 14D, lanes 3 and 4). Surprisingly, 5′ppp-SEQ ID NO: 1 did not further increase the level of phospho-IRF3 compared to DENV infection alone (FIG. 14D, lane 3 and 4), an observation that is in part attributable to the early and transient kinetics of IRF3 phosphorylation. These data demonstrate that RIG-I activation by 5′ppp-SEQ ID NO: 1 triggers an immune response capable of inhibiting DENV in both primary and ADE models of infection.

### Example 1

### 5′ppp-SEQ ID NO: 1 Treatment Inhibits CHIKV Replication in a RIG-1-Dependent Manner

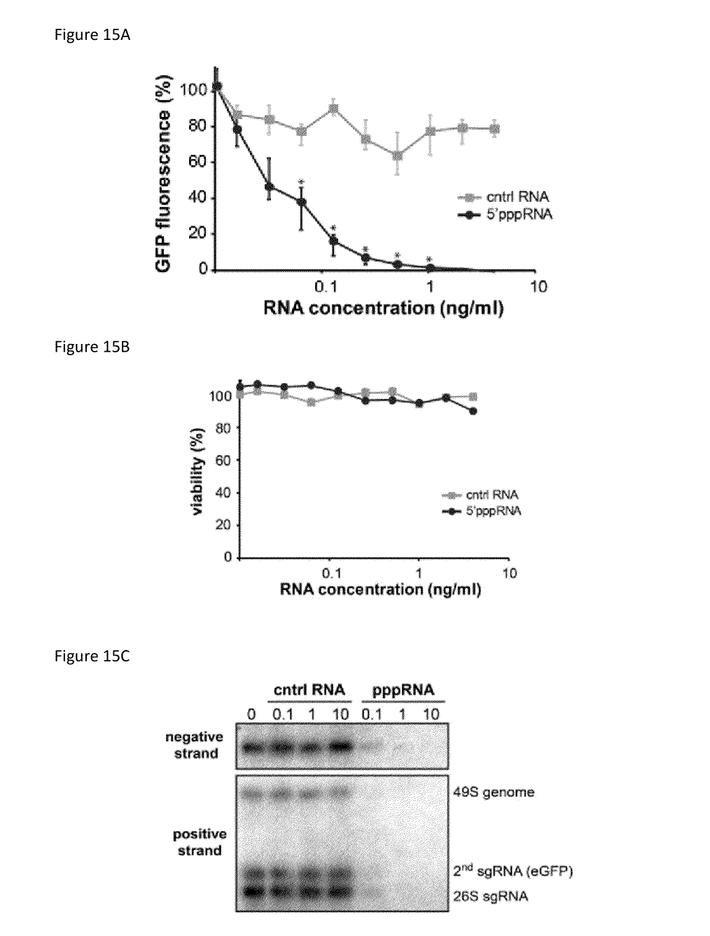

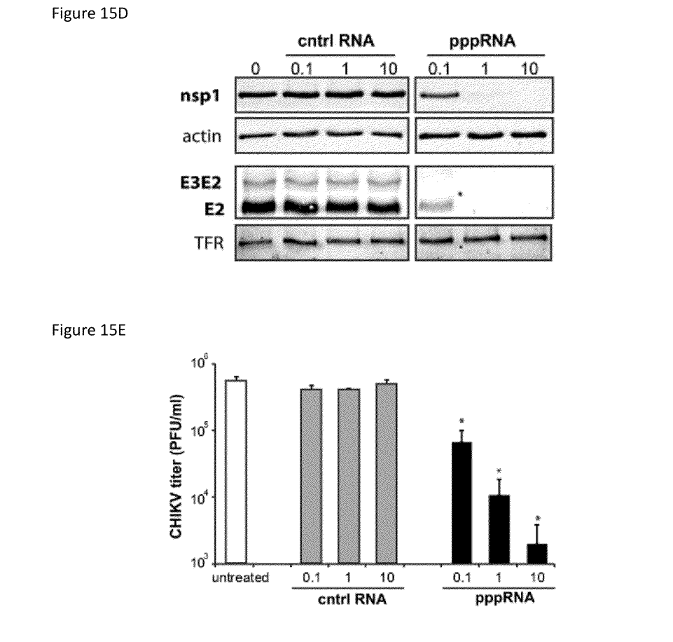

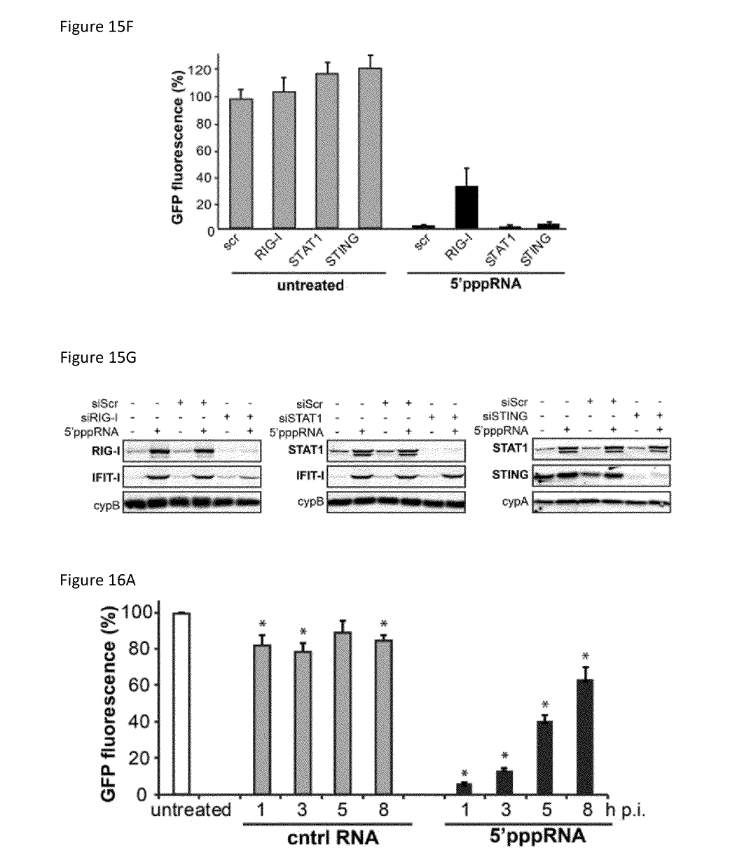

To explore the potential of 5′ppp-SEQ ID NO: 1 to prevent CHIKV infection, human fibroblast MRC-5 cells were pretreated with increasing concentrations of 5′ppp-SEQ ID NO: 1 prior to challenge with a CHIKV LS3-GFP reporter virus (FIG. 15A). CHIKV replication was strongly inhibited in a dose-dependent manner in cells treated with 5′ppp-SEQ ID NO: 1 one hour prior to infection (FIG. 15A); as little as 1 ng/ml completely blocked CHIKV EGFP reporter gene expression, and the 5′ppp-SEQ ID NO: 1 concentration required to completely block CHIKV replication in MRC-5 cells was 10-fold lower than that required to inhibit DENV in A549 cells. It is currently unclear whether this is due to virus-specific immune evasion or cell type-specific differences, as CHIKV does not replicate in A549 cells. Also, introduction of control RNA lacking the 5′-triphosphate moiety only led to a minor reduction of GFP reporter gene expression in CHIKV LS3-GFP-infected cells (FIG. 15A). Cell viability, monitored in parallel, was not significantly affected by transfection of either 5′ppp-SEQ ID NO: 1 or control RNA lacking the 5′ triphosphate (FIG. 15B). Analysis of intracellular RNA of CHIKV-infected cells pretreated 5′ppp-SEQ ID NO: 1 or control RNA showed that treatment with 0.1 ng/ml 5′ppp-SEQ ID NO: 1 reduced CHIKV positive- and negative-strand RNA accumulation to minimally detectable levels (FIG. 15C), and at higher doses of 5′ppp-SEQ ID NO: 1 was undetectable. Transfection of cells with control RNA prior to infection had no significant effect on the accumulation of CHIKV RNA (FIG. 15C). To determine the effect 5′ppp-SEQ ID NO: 1 treatment on the expression of CHIKV nonstructural proteins (translated from genomic RNA) and structural proteins (translated from the sgRNA), cells were pretreated with 5′ppp-SEQ ID NO: 1 or control RNA and infected with CHIKV, and nsP1 and E2 expression was analyzed by Western blotting (FIG. 15D). Transfection of 0.1 ng/ml 5′ ppp-SEQ ID NO: 1 led to a 4-fold reduction in nsP1 expression and an 8-fold reduction in E2 expression. Higher doses of 5′ppp-SEQ ID NO: 1 reduced nsP1 and E2 expression over 30-fold (FIG. 15D). Transfection of control RNA lacking the 5′ triphosphate had no noticeable effect on CHIKV protein expression (FIG. 15D). Finally, the effect of 5′ppp-SEQ ID NO: 1 treatment on the production of infectious progeny was determined. Compared to untreated cells, transfection of MRC-5 cells with 0.1 ng/ml of 5′ppp-SEQ ID NO: 1 one hour prior to CHIKV infection led to a 1 log reduction in virus titer, while transfection with 1 ng/ml and 10 ng/ml 5′ppp-SEQ ID NO: 1 reduced viral progeny titers by 2 and 3 logs, respectively (FIG. 15E). Transfection of control RNA lacking the 5′ triphosphate did not significantly affect CHIKV progeny titers (FIG. 15E).

To determine which innate immune pathways are involved in the 5′ppp-SEQ ID NO: 1 mediated inhibition of CHIKV replication, several key proteins of the IFN signaling pathway (RIG-I, STAT1, and STING) were depleted in MRC-5 cells using siRNAs. Knockdown levels were assessed by Western blotting (FIG. 15G). Subsequently, cells depleted for RIG-I, STAT1, or STING were treated with 5′ppp-SEQ ID NO: 1 and infected 1 h later with CHIKV LS3-GFP (FIG. 15F). CHIKV-driven GFP reporter gene activity was reduced to almost background levels in 5′ppp-SEQ ID NO: 1-treated cells that were depleted for STAT1 and STING, suggesting these proteins are not involved in the 5′ppp-SEQ ID NO: 1-mediated antiviral response to CHIKV. In contrast, CHIKV replication was observed in cells depleted of RIG-I and treated with 5′ppp-SEQ ID NO: 1, although EGFP reporter gene expression was 30% of that in untreated cells transfected with scrambled (or RIG-1-targeting) siRNAs (FIG. 15F). This partial recovery of replication might be due to incomplete knockdown of RIG-I in a fraction of the cells and/or paracrine IFN signaling of those cells, which could affect CHIKV replication of RIG-1-depleted cells. CHIKV replication in cells depleted for RIG-I, STAT1, or STING, but not treated with 5′ppp-SEQ ID NO: 1, was similar or slightly increased compared to that of cells transfected with a scrambled control siRNA. In parallel, the siRNA-treated cells were transfected with 1 ng/ml 5′ppp-SEQ ID NO: 1, and 24 h later the IFN signaling response was analyzed by monitoring the upregulation of IFIT-I or STAT1 (FIG. 15G). Knockdown of RIG-I expression resulted in a strong reduction of 5′ppp-SEQ ID NO: 1-induced IFIT-I upregulation, whereas the 5′ppp-SEQ ID NO: 1—induced upregulation of IFIT-I was not affected by STAT-1 depletion. siRNA-mediated knockdown of STING also did not block the 5′ppp-SEQ ID NO: 1—induced upregulation of STAT1, indicating that STAT1 and STING are dispensable for the response to 5′ppp-SEQ ID NO: 1, whereas RIG-I is required.

### Example 13

### Postinfection Treatment with 5′Ppp-SEQ ID NO: 1 Inhibits CHIKV Replication and Stimulates the RIG-I Pathway in Both Uninfected and CHIKV-Infected Cells

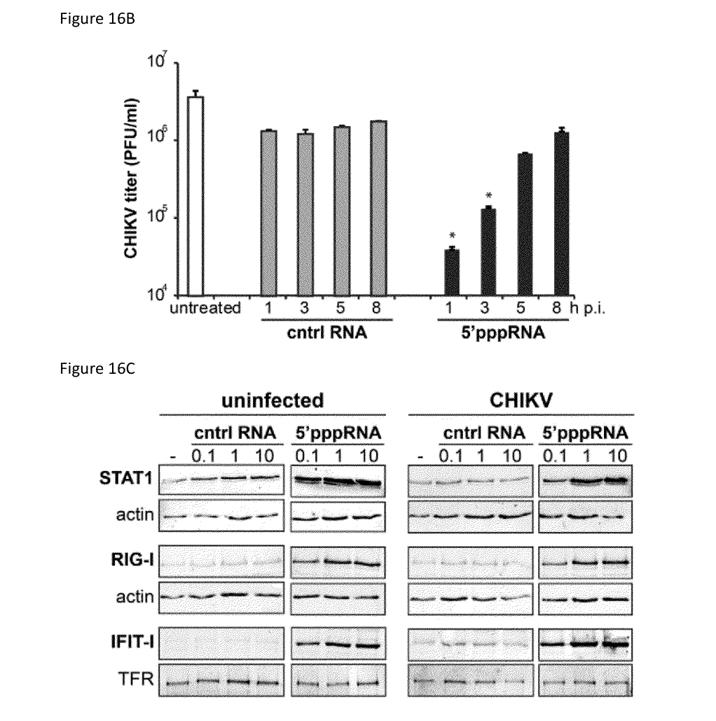

To explore the antiviral potential of 5′ppp-SEQ ID NO: 1 against CHIKV, MRC-5 cells were first infected with CHIKV LS3-GFP at an MOI of 0.1, followed by transfection with 5′ppp-SEQ ID NO: 1 (1 ng/ml) or control RNA at several time points postinfection. Measurement of EGFP expression by the reporter virus in infected MRC-5 cells that were fixed at 24 h p.i. indicated that treatment with 5′ppp-SEQ ID NO: 1 at 1 or 3 h p.i. reduced reporter gene expression to less than 20% of that in untreated infected control cells (FIG. 16A). Even when treatment was initiated as late as 5 h p.i., a more than 50% reduction in EGFP expression was observed (FIG. 16A). Transfection of control RNA merely led to a 20% reduction in EGFP reporter gene expression, largely independent of the time of addition. Postinfection treatment of CHIKV-infected cells with 5′ppp-SEQ ID NO: 1 also reduced viral progeny titers at 24 h p.i., depending on the time of addition (FIG. 16B). CHIKV titers in the medium of untreated infected cells were 6×10⁶ PFU/ml at 24 h p.i., while treatment from 1 h p.i. onward led to a more than 2-log reduction in infectious progeny, i.e., 5×10⁴ PFU/ml. When treatment was initiated at 3, 5, or 8 h p.i., CHIKV titers of 2×10⁵, 7×10⁵, and 1×10⁶, respectively, were measured at 24 h p.i. Transfection of CHIKV-infected cells with control RNA resulted in a less than 1-log reduction in infectious progeny titer (FIG. 16B).

To assess the activation of the RIG-I signaling pathway in MRC-5 cells after 5′ppp-SEQ ID NO: 1 treatment in the presence or absence of CHIKV infection, the expression levels of STAT1, RIG-I, and IFIT1 were analyzed by immunoblotting (FIG. 16C). Both in mock infected and CHIKV-infected cells, transfection of 0.1 ng/ml 5′ppp-SEQ ID NO: 1 induced a strong upregulation of STAT1, RIG-I, and IFIT-I (FIG. 16C), an effect that was more pronounced with treatment of 1 or 10 ng/ml of 5′ppp-SEQ ID NO: 1. In contrast, introduction of control RNA had no effect on expression of these proteins. CHIKV infection alone did not lead to increased STAT1, RIG-I, and IFIT1 expression, and CHIKV infection did not inhibit the 5′ppp-SEQ ID NO: 1-induced upregulation of RIG-I or downstream IFN signaling (FIG. 16C).

### Example 14

### Materials and Methods

Materials and Methods in this Example are in Reference to Examples 8-13 Above.

In vitro Synthesis of 5′ppp-SEQ ID NO: 1.

The sequence of 5′ppp-SEQ ID NO: 1 was derived from the 5′ and 3′ untranslated regions (UTR) of the VSV genome as described above. In vitro-transcribed RNA was prepared as described above and in Goulet M L et al, PLoS Pathol 9, e1003298 (2013), which is incorporated by reference herein. RNA was prepared using the Ambion MEGAscript T7 kit according to the manufacturer's guidelines (Invitrogen, NY, USA). 5′ppp-SEQ ID NO: 1 was purified using the Qiagen miRNA minikit (Qiagen, Valencia, Calif.). An RNA with the same sequence but lacking the 5′ ppp moiety was purchased from IDT (Integrated DNA Technologies Inc., IA, USA). This RNA generated results identical to those obtained with 5′ppp-SEQ ID NO: 1 that was dephosphorylated enzymatically with calf intestinal alkaline phosphatase (Invitrogen, NY, USA).

Cell Culture and Transfections.

A549 cells were grown in F12K medium (ATCC, Manassas, Va.) supplemented with 10% fetal bovine serum (FBS) and antibiotics. C6/36 insect cells were cultured in Dulbecco's modified Eagle medium (DMEM) supplemented with 10% FBS and antibiotics. Lipofectamine RNAiMax (Invitrogen, NY, USA) was used for transfections of 5′ppp-SEQ ID NO: 1 in A549 cells according to the manufacturer's instructions. For short interfering RNA (siRNA) knockdown, A549 cells were transfected with 50 nM (30 pmol) human RIG-I (sc-6180), IFN-α/βR α chain (sc-35637) and β chain (sc-40091), STING (sc-92042), TLR3 (sc-36685), MDA5 (sc-61010), MAVS (sc-75755), interleukin-28R (IL-28R; sc-62497), IL-10R

(sc-75331), STAT1 p844/91 (sc-44123), IRF1 (sc-35706), IRF3 (sc-35710), IRF7 (sc-38011), and control siRNA (sc-37007) (Santa Cruz Biotechnology, Dallas, T) using Lipofectamine RNAiMax according to the manufacturer's guidelines.

MRC-5 cells (ATCC CCL-171) were grown in Earle's minimum essential medium (EMEM) supplemented with 10% FBS, 2 mM L-glutamine, 1% nonessential amino acids (PAA), and antibiotics. For siRNA mediated knockdown of gene expression, MRC-5 cells were transfected with 16.7 nM (10 pmol) siRNA using Dharmafect1 (Dharmacon) according to the manufacturer's guidelines. Mouse embryonic fibroblast cells (MEFs) were grown in DMEM with 10% FBS and antibiotics.

Primary Cell Isolation.

Human peripheral blood mononuclear cells (PBMC) were isolated from the blood of healthy volunteers in a study approved by the institutional review board and by the VGTI-FL Institutional Biosafety Committee (2011-6-JH1). Written informed consent, approved by the VGTI-FL Inc. ethics review board (FWA number 161), was provided and signed by study participants. Research conformed to ethical guidelines established by the ethics committee of the OHSU VGTI and Martin Health System. Briefly, PBMC were isolated from freshly collected blood using Ficoll-Paque plus medium (GE Healthcare Bio, Uppsala, Sweden) per the manufacturer's instructions. Monocytes were then isolated using the negative selection human monocyte enrichment kit (Stem Cell, Vancouver, Canada) per the kit's instructions and used for further experiments. To obtain monocyte-derived dendritic cells (MDDC), monocytes were allowed to adhere to 100-mm dishes for 1 h inserum-free RPMI at 37° C. After adherence, remaining platelets and nonadherent cells were removed by two washes with serum-free RPMI. The cells were differentiated into MDDC by culturing for 7 days in Mo-DC differentiation medium (Miltenyi Biotec, Auburn, Ga.). Medium was replenished after 3 days of differentiation.

Virus Production, Quantification, and Infection.

Confluent monolayers of C6/36 insect cells were infected with DENV serotype 2 strain New Guinea C (DENV NGC) at a multiplicity of infection (MOI) of 0.5. Virus was allowed to adsorb for 1 h at 28° C. in a minimal volume of serum-free DMEM. After adsorption, the monolayer was washed once with serum free medium and covered with DMEM containing 2% FBS. After 7 days of infection, medium was harvested, cleared by centrifugation (500×g, 5 min), and concentrated down by centrifugation (2,000×g, 8 min) through a 15-ml Millipore Amicon centrifugal filter unit (Millipore, Billerica, Mass.). The virus was concentrated by ultracentrifugation on a sucrose density gradient (20% sucrose cushion) using a Sorvall WX 100 ultracentrifuge (ThermoScientific, Rockford, Ill.) for 2 h at 134,000×g and 10° C. with the brake turned off. Concentrated virus was then washed to remove sucrose using a 15-ml Amicon tube. After 2 washes, the virus was resuspended in DMEM plus 0.1% bovine serum albumin (BSA) and stored at −80° C. Titers of DENV stocks were determined by fluorescence activated cell sorting (FACS), infecting Vero cells with 10-fold serial dilutions of the stock, and then immunofluorescence staining of intracellular DENV E protein at 24 h postinfection (p.i.). Titers were expressed as IU/ml. DENV titers in cell culture supernatants from 5′ppp-SEQ ID NO: 1-treated and control cells were determined by plaque assay on confluent Vero cells. Cells in 6-well clusters were incubated with 10-fold serial dilutions of the sample in a total volume of 500 μl of DMEM without serum. After 1 h of infection, the inoculum was removed and cells were overlaid with 3 ml of 2% agarose in complete DMEM. The cells were fixed and stained, and plaques were counted 5 days postinfection.

In infection experiments, A549 cells, monocytes, or MDDC were infected in a small volume of medium without FBS for 1 h at 37° C. and then incubated with complete medium for 24 to 72 h prior to analysis. All procedures with live DENV were performed in a biosafety level 2

facility at the Vaccine and Gene Therapy Institute-Florida.

Chikungunya virus (CHIKV) strain LS3 and enhanced green fluorescent protein (EGFP)-expressing reporter virus CHIKV LS3-GFP have been described (Scholte F E et al, PLoS One 8, e71047 (2013); incorporated by reference herein). Virus production, titration, and infection were performed essentially as described in the art. Working stocks of CHIKV were routinely produced in Vero E6 cells at 37° C., and infections were performed in EMEM with 25 mM HEPES (Lonza) supplemented with 2% fetal calf serum (FCS), L-glutamine, and antibiotics. After 1 h, the inoculum was replaced with fresh culture medium. All procedures with live CHIKV were performed in a biosafety level 3 facility at the Leiden University Medical Center.

Flow Cytometry Analysis.

The percentage of cells infected with DENV was determined by standard intracellular staining (ICS) with a mouse IgG2a monoclonal antibody (MAb) specific for DENV-E protein (clone 4G2), followed by staining with a secondary anti-mouse antibody coupled to phycoerythrin (PE) (BioLegend, San Diego, Calif.). Cells were analyzed on an LSRII flow cytometer (Becton, Dickinson, N.J., USA). Calculations as well as population analyses were done using FACS Diva software.

Cell Viability Analysis.

Cell surface expression of phosphatidylserine was measured using an allophycocyanin (APC)-conjugated annexin V antibody, as recommended by the manufacturer (BioLegend, San Diego, Calif.). Briefly, specific annexin V binding was achieved by incubating A549 cells in annexin V binding buffer (Becton, Dickinson, N.J., USA) containing a saturating concentration of APC-annexin V antibody and 7-aminoactinomycin D (7-AAD) (Becton, Dickinson, N.J., USA) for 15 min in the dark. APC-annexin V and 7-AAD binding to the cells was analyzed by flow cytometry, as described previously, using an LSRII flow cytometer and FACS Diva software. Alternatively, the viability of siRNA or 5′ppp-SEQ ID NO: 1—transfected cells was assessed using the CellTiter 96 aqueous nonradioactive cell proliferation assay (Promega). Absorbance was measured using a Berthold Mithras LB 940 96-well plate reader.

Protein Extraction and Immunoblot Analysis.

DENV-infected cells were washed twice in ice-cold phosphate-buffered saline (PBS) and lysed in radioimmunoprecipitation assay (RIPA) buffer (50 mN Tris-HCl, pH 8, 1% sodium deoxycholate, 1% NP-40, 5 mM EDTA, 150 mM NaCl, 0.1% sodium dodecyl sulfate), and the insoluble fraction was removed by centrifugation at 17,000 g for 15 min (4° C.). Protein concentration was determined using the Pierce bicinchoninic (BCA) protein assay kit (Thermo Scientific, Rockford, Ill.). Protein extracts were resolved by SDS-PAGE on 4 to 20% acrylamide Mini-Protean TGX precast gels (Bio-Rad, Hercules, Calif.) in a 1 Tris-glycine-SDS buffer (Bio-Rad, Hercules, Calif.). Proteins were electrophoretically transferred to an Immobilon-PSQ polyvinylidene difluoride (PVDF) membrane (Millipore, Billerica, Mass.) for 1 h at 100 V in a buffer containing 30 mM Tris, 200 mM glycine, and 20% methanol. Membranes were blocked for 1 h at room temperature in Odyssey blocking buffer (Odyssey, USA) and then probed with the following primary antibodies: anti-IRF1 (Santa Cruz Biotechnology, Dallas, Tex.), anti-pIRF3 at Ser 396 (EMD Millipore, MA, USA), anti-IRF3 (IBL, Japan), anti-IRF7 (Cell Signaling, MA, USA), anti-RIG-I (EMD Millipore, MA, USA), anti-IFIT1 (Thermo Fisher Scientific, Rockford, Ill., USA), anti-ISG15 (Cell Signaling Technology, Danvers, Mass.), anti-pSTAT1 at Tyr701 (Cell Signaling, MA, USA), anti-STAT1 (Cell Signaling, MA, USA), anti-STING (Novus Biologicals, Littleton, Colo.), anti-DENV (Santa Cruz Biotechnology, USA), and anti-actin (Odyssey, USA). Antibody signals were detected by immunofluorescence using the IRDye 800CW and IRDye 680RD secondary antibodies (Odyssey, USA) and the LiCor imager (Odyssey, USA). Protein expression levels were determined and normalized to β-actin using ImageJ software (National Institutes of Health, Bethesda, Md.).

CHIKV-infected cells were lysed and proteins were analyzed by Western blotting. CHIKV proteins were detected with rabbit antisera against nsP1 (a generous gift of Andres Merits, University of Tartu, Estonia) and E2 (Aguirre S, PLos Pathog 8, 31002934 (2012); incorporated by reference herein). Mouse monoclonal antibodies against β-actin (Sigma), the transferrin receptor (Zymed), cyclophilin A (Abcam), and cyclophilin B (Abcam) were used for detection of loading controls. Biotin-conjugated swine α-rabbit (Dako), goat α-mouse (Dako), and Cy3-conjugated mouse α-biotin (Jackson) were used for fluorescent detection of the primary antibodies with a Typhoon-9410 scanner (GE Healthcare).

RT-qPCR.

Total RNA was isolated from cells using an RNeasy kit (Qiagen, Valencia, Calif.) per the manufacturer's instructions. RNA was reverse transcribed using the SuperScript VILO cDNA synthesis kit according to the manufacturer's instructions (Invitrogen, Carlsbad, Calif.). PCR primers were designed using Roche's Universal Probe Library Assay Design Center (Roche). Quantitative reverse transcription-PCR (RTqPCR) was performed on a LightCycler 480 system using LightCycler 480 probes master (Roche, Penzberg, Germany). All data are presented as a relative quantification with efficiency correction based on the relative expression of target gene versus glyceraldehyde-3-phosphate dehydrogenase (GAPDH) as the invariant control. The N-fold differential mRNA expression of genes in samples was expressed as 2^{ΔΔCT}. Primers used are described in the Sequence Listing submitted with this application.

RNA Isolation, Denaturing Agarose Electrophoresis, and In-Gel Hybridization.

CHIKV RNA isolation and analysis were performed essentially as described in the art. Briefly, total RNA was isolated by lysis in 20 mM Tris-HCl (pH 7.4), 100 mM LiCl, 2 mM EDTA, 5 mM dithiothreitol (DTT), 5% (wt/vol) lithium dodecyl sulfate, and 100 μg/ml proteinase K. After acid phenol (Ambion) extraction, RNA was precipitated with isopropanol, washed with 75% ethanol, and dissolved in 1 mM sodium citrate (pH 6.4). RNA samples were separated in 1.5% denaturing formaldehyde-agarose gels using the morpholine propanesulfonic acid (MOPS) buffer system. RNA molecules were detected by direct hybridization of the dried gel with ³²P-labeled oligonucleotides. CHIKV genomic and subgenomic RNAs (sgRNAs) were visualized with probe CHIKV-hyb4 and negative-stranded RNA was detected with probe CHIKV-hyb2. Probes (10 pmol) were labeled with 10 μCi [γ-32P]ATP (PerkinElmer). Prehybridization (1 h) and hybridization (overnight) were done at 55° C. in 5×SSPE (0.9 M NaCl, 50 mM NaH2PO4, 5 mM EDTA, pH 7.4), 5×Denhardt's solution, 0.05% SDS, and 0.1 mg/ml homomix I. Storage Phosphor screens were exposed to hybridized gels and scanned with a Typhoon-9410 scanner (GE Healthcare), and data were quantified with Quantity One v4.5.1 (Bio-Rad).

Statistical Analysis.

Values were expressed as the means±standard errors of the means (SEM), and statistical analysis was performed with Microsoft Excel using an unpaired, two-tailed Student's t test to determine significance. Differences were considered significant at P<0.05.

1. A compound comprising
an oligoribonucleotide comprising a nucleic acid sequence of SEQ ID NO: 1; and
a triphosphate group covalently attached to the 5′ end of the oligoribonucleotide.

2. The compound of claim 1 wherein the oligoribonucleotide consists of SEQ ID NO: 1.

3. The compound of claim 1 wherein the oligoribonucleotide comprises a modified ribonucleotide.

4. The compound of claim 3 wherein the modified ribonucleotide comprises a 2′-O-methyl (2′OMe), 2′-deoxy-2′-fluoro (2′F), 2′-deoxy, 5-C-methyl, 2′-O-(2-methoxyethyl) (MOE), 4′-thio, 2′-amino, or 2′-C-allyl modification.

5. The compound of claim 3 wherein the modified ribonucleotide comprises a locked nucleic acid.

6. The compound of claim 5 wherein the locked nucleic acid is 2′-O, 4′-C-methylene-(D-ribofuranosyl)nucleotide, 2′-O-(2-methoxyethyl) (MOE) nucleotide, 2′-methyl-thio-ethyl nucleotide, 2′-deoxy-2′-fluoro (2′F) nucleotide, 2′-deoxy-2′-chloro (2Cl) nucleotide, or 2′-azido nucleotide.

7. The compound of claim 3 wherein the modified nucleotide comprises a G-clamp nucleotide.

8. The compound of claim 3 wherein the modified nucleotide comprises a nucleotide base analog.

9. The compound of claim 8 wherein the nucleotide base analog comprises C-phenyl, C-naphthyl, inosine, azole carboxamide, or nitroazole.

10. The compound of claim 9 wherein the moiety is nitroazole and is 3-nitropyrrole, 4-nitroindole, 5-nitroindole, or 6-nitroindole.

11. The compound of claim 1 comprising a 3′ terminal cap moiety.

12. The compound of claim 11 wherein the terminal cap moiety is an inverted deoxy abasic residue, a glyceryl modification, a 4′,5′-methylene nucleotide, a 1-(β-D-erythrofuranosyl) nucleotide, a 4′-thio nucleotides, carbocyclic nucleotide, a 1, 5-anhydrohexitol nucleotide, an L-nucleotide, an α-nucleotide, a modified base nucleotide, a threo pentofuranosyl nucleotide, an acyclic 3′,4′-seco nucleotide, an acyclic 3,4-dihydroxybutyl nucleotide, an acyclic 3,5-dihydroxypentyl nucleotide, a 3′-3′-inverted nucleotide moiety, a 3′-3′-inverted abasic moiety, a 3′-2′-inverted nucleotide moiety, a 3′-2′-inverted abasic moiety, a 5′-5′-inverted nucleotide moiety, a 5′-5′-inverted abasic moiety, a 3′-5′-inverted deoxy abasic moiety, a 5′-amino-alkyl phosphate, a 1,3-diamino-2-propyl phosphate, a 3-aminopropyl phosphate, a 6-aminohexyl phosphate, a 1,2-aminododecyl phosphate, a hydroxypropyl phosphate, a 1,4-butanediol phosphate, a 3′-phosphoramidate, a 5′-phosphoramidate, a hexylphosphate, an aminohexyl phosphate, a 3′-phosphate, a 5′-amino, 3′-phosphorothioate, a 5′-phosphorothioate, a phosphorodithioate, a bridging methylphosphonate, a non-bridging methylphosphonate, or a 5′-mercapto group.

13. The compound of claim 1 wherein the oligoribonucleotide comprises a phosphate backbone modification.

14. The compound of claim 13 wherein the phosphate backbone modification is a phosphorothioate, phosphorodithioate, methylphosphonate, phosphotriester, morpholino, amidate, carbamate, carboxymethyl, acetamidate, polyamide, sulfonate, sulfonamide, sulfamate, formacetal, thioformacetal, or alkylsilyl substitution.

15. The compound of claim 1 further comprising a conjugate attached to the oligoribonucleotide.

16. The compound of claim 15 wherein the conjugate is attached to the 3′ end of the oligoribonucleotide.

17. A pharmaceutical composition comprising a therapeutically effective amount of the compound of claim 1 and a pharmaceutically acceptable carrier.

18. The pharmaceutical composition of claim 17 wherein the pharmaceutically acceptable carrier acts as a transfection reagent.

19. The pharmaceutical composition of claim 18 wherein the pharmaceutically acceptable carrier comprises a lipid based carrier, a polymer based carrier, a cyclodextrin based carrier, or a protein based carriers.

20. The pharmaceutical composition of claim 19 wherein the pharmaceutically acceptable carrier is a lipid based carrier comprising a stabilized nucleic acid-lipid particle, a cationic lipid, a liposome nucleic acid complex, a liposome, a micelle, or a virosome.

21. A method of treating a viral infection in a subject, the method comprising:
administering the pharmaceutical composition of claim 17 to the subject.

22. The method of claim 21 wherein the viral infection is caused by vesicular stomatitis virus, dengue virus, vaccinia virus, human immunodeficiency virus, chikungunya virus, or influenza virus.

23. The method of claim 20 wherein the pharmaceutical composition is administered prophylactically or therapeutically.

24. The method of claim 20 wherein the route of administration is, oral, sublingual, rectal, transdermal, intranasal, vaginal, retro-orbital, by inhalation, or by injection.

25. The method of claim 24 wherein the route of administration is by injection and wherein the mode of injection is subcutaneous, intramuscular, intradermal, intraperitoneal, or intravenous.


SEQUENCE LISTING (54 sequences)

SEQ ID NO: 1 - RNA, 67 nt, Artificial Sequence
  Oligoribonucleotide derived from VSV
gacgaagaccacaaaaccagauaaaaaauaaaauuuuaaugauaauaaugguuuguuugucuucguc

SEQ ID NO: 2 - DNA, 178 bp, Artificial Sequence
  DNA template derived from VSV
gacgaagacaaacaaaccattattatcattaaaattttattttttatctggttttgtggtcttcgtctatagtgagtcgtattaatttcgaaattaatacgactcactatagacgaagaccacaaaaccagataaaaaataaaattttaatgataataatggtttgtttgtcttcgtc

SEQ ID NO: 3 - DNA, 20 bp, Artificial Sequence
  IFNB1 Forward Primer
ttgtgcttctccactacagc

SEQ ID NO: 4 - DNA, 20 bp, Artificial Sequence
  IFNB1 reverse primer
ctgtaagtctgttaatgaag

SEQ ID NO: 5 - DNA, 21 bp, Artificial Sequence
  IL29 forward primer
ggacgccttggaagagtcact

SEQ ID NO: 6 - DNA, 21 bp, Artificial Sequence
  IL29 reverse primer
agaagcctcaggtcccaattc

SEQ ID NO: 7 - DNA, 20 bp, Artificial Sequence
  IRF7 forward primer
cttcgtgatgctgcgagata

SEQ ID NO: 8 - DNA, 20 bp, Artificial Sequence
  IRF7 reverse primer
aagcccttcttgtccctctc

SEQ ID NO: 9 - DNA, 20 bp, Artificial Sequence
  CCL5 forward primer
ctttgtcacccgaaagaacc

SEQ ID NO: 10 - DNA, 20 bp, Artificial Sequence
  CCL5 reverse primer
ctgtaagtctgttaatgaag

SEQ ID NO: 11 - DNA, 25 bp, Artificial Sequence
  CXCL10 forward primer
tcttctcacccttctttttcattgt

SEQ ID NO: 12 - DNA, 21 bp, Artificial Sequence
  CXCL10 reverse primer
ttcctgcaagccaattttgtc

SEQ ID NO: 13 - DNA, 19 bp, Artificial Sequence
  IL6 forward primer
ggagacttcctggtgaaaa

SEQ ID NO: 14 - DNA, 20 bp, Artificial Sequence
  IL6 reverse primer
atctgaggtgcccatgctac

SEQ ID NO: 15 - DNA, 19 bp, Artificial Sequence
  ISG15 forward primer
agctccatgtcggtgtcag

SEQ ID NO: 16 - DNA, 20 bp, Artificial Sequence
  ISG15 reverse primer
gaaggtcagccagaacaggt

SEQ ID NO: 17 - DNA, 20 bp, Artificial Sequence
  ISG56 forward primer
caaccaagcaaatgtgagga

SEQ ID NO: 18 - DNA, 20 bp, Artificial Sequence
  ISG56 reverse primer
aggggaagcaaagaaaatgg

SEQ ID NO: 19 - DNA, 17 bp, Artificial Sequence
  RIG-I forward primer
gcagaggccggcatgac

SEQ ID NO: 20 - DNA, 20 bp, Artificial Sequence
  RIG-I reverse primer
aatcccatcaccatcttcca

SEQ ID NO: 21 - DNA, 23 bp, Artificial Sequence
  Viperine foward primer
cacaaagaagtgtcctgcttggt

SEQ ID NO: 22 - DNA, 27 bp, Artificial Sequence
  Viperine reverse primer
aagcgcatatatttcatccagaataag

SEQ ID NO: 23 - DNA, 24 bp, Artificial Sequence
  OASL forward primer
ggatcttctcccacactcacatct

SEQ ID NO: 24 - DNA, 22 bp, Artificial Sequence
  OASL reverse primer
caccatcaggattcttcacgaa

SEQ ID NO: 25 - DNA, 20 bp, Artificial Sequence
  NOXA forward primer
agctggaagtcgagtgtgct

SEQ ID NO: 26 - DNA, 21 bp, Artificial Sequence
  NOXA reverse primer
tcctgagcagaagagtttgga

SEQ ID NO: 27 - DNA, 20 bp, Artificial Sequence
  GADPH forward primer
aatcccatcaccatcttcca

SEQ ID NO: 28 - DNA, 20 bp, Artificial Sequence
  GADPH reverse primer
tgagtccttccacgatacca

SEQ ID NO: 29 - DNA, 20 bp, Artificial Sequence
  Dengue virus forward primer
caaggcgagatgaagctgta

SEQ ID NO: 30 - DNA, 20 bp, Artificial Sequence
  Dengue virus reverse primer
ggtctttcccagcgtcaata

SEQ ID NO: 31 - DNA, 22 bp, Artificial Sequence
  PCR primer
atcctcctatggtacgcacaaa

SEQ ID NO: 32 - DNA, 26 bp, Artificial Sequence
  PCR Primer
ctccagtattattgaagctgctatcc

SEQ ID NO: 33 - DNA, 19 bp, Artificial Sequence
  PCR Primer
agccacatcgctcagacac

SEQ ID NO: 34 - DNA, 19 bp, Artificial Sequence
  PCR Primer
gcccaatacgaccaaatcc

SEQ ID NO: 35 - DNA, 20 bp, Artificial Sequence
  PCR Primer
aatggccttgacctttgctt

SEQ ID NO: 36 - DNA, 20 bp, Artificial Sequence
  PCR Primer
cacagagcagcttgacttgc

SEQ ID NO: 37 - DNA, 21 bp, Artificial Sequence
  PCR Primer
atttacaccatttcgcaaagc

SEQ ID NO: 38 - DNA, 26 bp, Artificial Sequence
  PCR Primer
cactattgccttatcttcagcttcta

SEQ ID NO: 39 - DNA, 20 bp, Artificial Sequence
  PCR Primer
tagcctccccaaagtcttga

SEQ ID NO: 40 - DNA, 21 bp, Artificial Sequence
  PCR Primer
aaatgacctccaccatatcca

SEQ ID NO: 41 - DNA, 25 bp, Artificial Sequence
  PCR Primer
ctttgctattttcagacaagattca

SEQ ID NO: 42 - DNA, 20 bp, Artificial Sequence
  PCR Primer
gccaggaggttctcaacaat

SEQ ID NO: 43 - DNA, 20 bp, Artificial Sequence
  PCR Primer
tgacgccctcaatcaaagta

SEQ ID NO: 44 - DNA, 23 bp, Artificial Sequence
  PCR Primer
tgacttataagcacccatgtcaa

SEQ ID NO: 45 - DNA, 20 bp, Artificial Sequence
  PCR Primer
caggagcccagctatgaact

SEQ ID NO: 46 - DNA, 18 bp, Artificial Sequence
  PCR Primer
gaaggcagcaggcaacac

SEQ ID NO: 47 - DNA, 20 bp, Artificial Sequence
  PCR Primer
cccccactggatctgaagta

SEQ ID NO: 48 - DNA, 23 bp, Artificial Sequence
  PCR Primer
gagtgactggaaatagggtcttg

SEQ ID NO: 49 - DNA, 19 bp, Artificial Sequence
  PCR Primer
cctgaggcttctccaggtg

SEQ ID NO: 50 - DNA, 17 bp, Artificial Sequence
  PCR Primer
ccaggaccttcagcgtc

SEQ ID NO: 51 - DNA, 20 bp, Artificial Sequence
  PCR Primer
gacaagcctgtagcccatgt

SEQ ID NO: 52 - DNA, 17 bp, Artificial Sequence
  PCR Primer
ctcagctccacgccatt

SEQ ID NO: 53 - DNA, 28 bp, Artificial Sequence
  Oligonucleotide probe
tgtgggttcggagaatcgtggaagagtt

SEQ ID NO: 54 - DNA, 28 bp, Artificial Sequence
  Oligonucleotide probe
aacccatcatggatcctgtgtacgtgga